(19) United States
(12) Patent Application Publication
(10) Pub. No.: US 2012/0316240 A1
(43) Pub. Date: Dec. 13, 2012

(54) METHOD OF DIAGNOSIS

(75) Inventors: Shaochun Song, Vista, CA (US); Yun Liu, San Diego, CA (US); Nan Jiang, Vista, CA (US); Lei Yu, Carlsbad, CA (US)

(73) Assignee: NITTO DENKO CORPORATION, Osaka (JP)

(21) Appl. No.: 13/324,750

(22) Filed: Dec. 13, 2011

Related U.S. Application Data
(60) Provisional application No. 61/425,502, filed on Dec. 21, 2010.

(51) Int. Cl. C40B 30/04; A61P 1/16; C12Q 1/02; C12Q 1/68; A61K 31/203

(52) U.S. Cl. 514/559; 506/9; 435/6.12; 435/29

(57) ABSTRACT

Disclosed herein are methods that generally to the fields of pharmaceutical chemistry, biochemistry, molecular biology and medicine. More particularly, aspects of the invention concern methods to identify a predisposition for fibrosis in a biological sample, methods of identifying agents that modulate the onset of fibrosis, methods of making a formulation that inhibits the onset or progression of fibrosis, and methods of monitoring the progression of fibrosis or the efficacy of a fibrosis treatment.

### CROSS-REFERENCE TO RELATED APPLICATIONS

The present application claims priority under 35 U.S.C. §119(e) to U.S. Provisional Patent Application No. 61/425,502, filed Dec. 21, 2010, the content of which is hereby expressly incorporated by reference in its entirety.

### FIELD OF THE INVENTION

Disclosed herein are methods that generally relate to the fields of pharmaceutical chemistry, biochemistry, molecular biology and medicine. More particularly, aspects of the invention concern methods to identify a predisposition for fibrosis in a biological sample, methods of identifying agents that modulate the onset of fibrosis, methods of making a formulation that inhibits the onset or progression of fibrosis, and methods of monitoring the progression of fibrosis or the efficacy of a fibrosis treatment.

### BACKGROUND OF THE INVENTION

Fibrosis, or the development of excess fibrous connective tissue within the body, can occur in any organ and has been associated with disease and abnormal health conditions. Fibrosis can follow surgery in the form of adhesions, keloid tumors or hypertrophic scarring and can cause joint dislocation after a severe burn, wound or orthopaedic injury, for example. Several diseases can also contribute to fibrosis including, but not limited to, hepatitis, hypertension, tuberculosis, scleroderma, diabetes, atherosclerosis, pancreatitis, and cancer. That is, fibrosis is frequently a clinical manifestation of an underlying pathogenic infection or aberrant health condition.

Hepatic fibrosis, for example, can result from hepatitis infection, nonalcoholic steatohepatitis, malnutrition-related diabetes, parasites, tuberculosis, syphilis, and intrahepatic congestion resulting from heart disease or a disorder in the passage of bile. Hepatic stellate cells (HSCs) can become activated after liver injury and the activated HSCs can stimulate other cells to produce and secrete excessive amounts of extracellular matrix (ECM) proteins, including many different types of collagens and fibronectin, which is then deposited on interstitial tissue. In the final stages of hepatic fibrosis, hepatic cirrhosis can occur, which may result in hepatic failure and hepatocellular carcinoma.

Several investigators have tried to inhibit the onset of various forms of fibrosis. The discovery that activated T-lymphocytes and monocytes/macrophages can effective modulate fibroblasts and the production of fibrosis-forming collagen has led some to believe that interferons can be used to inhibit the onset of fibrosis (see, e.g., U.S. Pat. No. 5,312,621). Others have tried to inhibit the onset of fibrosis using a combination of angiotensin inhibitors and nitric oxide stimulators (see e.g., U.S. Pat. No. 6,139,847); benzoic hydrazide (see e.g., U.S. Pat. No. 5,374,660) or Transforming Growth Factor Beta (TGF-β) monomers (see e.g., U.S. Pat. Pub. No. 2009/0137,475); or Betaglycan (see e.g., U.S. Pat. No. 6,060,460).

Because treatment after the onset of fibrosis can be difficult, oftentimes involving transplantation, such as in the case of hepatic fibrosis, there is a tremendous need for early stage diagnosis. The diagnosis of fibrotic disease is usually done by performing a biopsy of the affected tissue. Tissue biopsy is a highly invasive technique, however, and may cause complications including infection, hemorrhage, pain, and injury of surrounding tissue. Accordingly, many researchers are investigating approaches to diagnose the onset of fibrosis using serum markers (see e.g., U.S. Pat. Nos. 7,244,619 and 7,141,380 and U.S. Pat. Pub. No. 2007/0178443) or biochemical markers such as hyaluronate and prothrombin (see e.g., Oberti et al., Gastroenterology. 1997; 113(5):1609-16). Other researchers have tried to employ various imaging techniques to diagnose the onset of fibrosis. For example, some approaches involve the correlation of analytic values of diffusion in MR images (Aube et al., J. Radiol. 2004; 85(3):301-6) or the detection of morphologic change in the liver by CT, MRI or ultrasonography (Kudo et al., Intervirology. 2008; 51 Suppl 1:17-26). Despite these advances, the need for more agents that inhibit the onset of fibrosis, in particular liver fibrosis, and approaches to detect the early onset of fibrosis is manifest.

### SUMMARY OF THE INVENTION

The present application concerns the discovery that the amount or level of expression of Transforming Growth Factor Beta receptor III (TGFbr3) or an RNA encoding TGFbr3 was significantly reduced in Hepatic stellate cells (HSCs) isolated from fibrotic livers as compared to the level or amount of TGFbr3 or an RNA encoding TGFbr3 detected in HSCs that were isolated from normal or non-fibrotic livers. Furthermore, it was observed that as the level of fibrosis of the liver increased over time, the amount of TGFbr3 or an RNA encoding TGFbr3 decreased. Experimental animal models of liver fibrosis were developed and the amount of TGFbr3 or an RNA encoding TGFbr3 was monitored over time by both microarray analysis and quantitative reverse transcriptase polymerase chain reaction (QRTPCR) analysis. As one of skill in the art readily appreciates, by detecting the amount of RNA encoding TGFbr3, one has indirectly detected the amount of TGFbr3 protein in a sample.

TGFbr3 expression levels were also normalized to the expression levels of an RNA encoding a housekeeping enzyme (e.g., actin or glyceraldehyde-3-phosphate dehydrogenase (GAPDH)). That is, the expression levels of TGFbr3 were analyzed by generating a fibrosis ratio that represents the amount of TGFbr3 detected in relation to the level of expression of an RNA encoding a housekeeping enzyme. Fibrosis ratios generated from such measurements on HSCs isolated from livers at various stages of fibrosis were analyzed and compared to fibrosis ratios generated from such measurements of HSCs isolated from normal or non-fibrotic livers. Accordingly, it was determined that the early onset of fibrosis can be identified, detected or diagnosed by monitoring the amount, level, or presence of TGFbr3 in isolated HSCs or the amount or level of expression of an RNA encoding TGFbr3 in isolated HSCs.

Accordingly, aspects of the invention include methods of identifying a predisposition for fibrosis in a tested subject (e.g., a human) comprising providing a first biological sample that comprises a first population HSCs obtained from said tested subject; isolating said first population of HSCs from said first biological sample; measuring the amount of TGFbr3 or an RNA encoding TGFbr3 in said first isolated population of HSCs; comparing the amount of TGFbr3 or an RNA encoding TGFbr3 measured in said first isolated population of HSCs to an amount of TGFbr3 or an RNA encoding TGFbr3 measured in a second population of isolated HSCs obtained from a second biological sample from a second subject; and identifying a predisposition for fibrosis in said tested subject when: (a) the amount of TGFbr3 or an RNA encoding TGFbr3 measured in said first isolated population of HSCs is less than the amount of TGFbr3 or an RNA encoding TGFbr3 measured in the second isolated population of HSCs obtained from said second biological sample from said second subject when said second subject does not have fibrosis; or (b) the amount of TGFbr3 or an RNA encoding TGFbr3 measured in said first isolated population of HSCs is less than or equal to the amount of TGFbr3 or an RNA encoding TGFbr3 measured in the second isolated population of HSCs obtained from said second biological sample from said second subject when said second subject has fibrosis. In some embodiments, the method above further comprises measuring the amount of a housekeeping enzyme (e.g., GAPDH, actin, or ubiquitin) or an RNA encoding said housekeeping enzyme in said first isolated population of HSCs and said second isolated population of HSCs.

Some of these methods further comprise combining a value representing the measured amount of TGFbr3 or an RNA encoding TGFbr3 in said first isolated population of HSCs and a value representing the measured amount of a housekeeping enzyme (e.g., GAPDH, actin, or ubiquitin) or an RNA encoding said housekeeping enzyme in said first isolated population of HSCs to obtain a first fibrosis score and comparing this first fibrosis score to a second fibrosis score obtained by combining a value representing the measured amount of TGFbr3 or an RNA encoding TGFbr3 in said second isolated population of HSCs and a value representing the measured amount of a housekeeping enzyme (e.g., GAPDH, actin, or ubiquitin) or an RNA encoding said housekeeping enzyme in said second isolated population of HSCs. By these approaches, the predisposition for fibrosis in said tested subject (e.g., a human) can be identified when: (a) the fibrosis score of the tested subject is less than the fibrosis score of said second subject when said second subject does not have fibrosis; and (b) the fibrosis score of the tested subject is equal to, or less than the fibrosis score of said second subject when said second subject has fibrosis.

The methods above can further comprise determining a ratio of the amount of a housekeeping enzyme (e.g., GAPDH, actin, or ubiquitin) or an RNA encoding said housekeeping enzyme and the amount of TGFbr3 or an RNA encoding TGFbr3 in said first population of isolated HSC and comparing this ratio to a ratio of the amount of a housekeeping enzyme (e.g., GAPDH, actin, or ubiquitin) or an RNA encoding said housekeeping enzyme and the amount of TGFbr3 or an RNA encoding TGFbr3 in said second isolated population of HSCs. In some of these methods, the predisposition of fibrosis in said tested subject is identified when a value representing the ratio of the amount of the housekeeping enzyme (e.g., GAPDH, actin beta or ubiquitin) or an RNA encoding said housekeeping enzyme and the amount of TGFbr3 or an RNA encoding TGFbr3 in said first isolated population HSCs is less than a value representing a ratio of the amount of the housekeeping enzyme (e.g., GAPDH, actin beta or ubiquitin) or an RNA encoding said housekeeping enzyme and the amount of TGFbr3 or an RNA encoding TGFbr3 in said second isolated population HSCs when said second subject does not have fibrosis. In other methods, the predisposition of fibrosis in said tested subject is identified when a value representing the ratio of the amount of the housekeeping enzyme (e.g., GAPDH, actin beta or ubiquitin) or an RNA encoding said housekeeping enzyme and the amount of TGFbr3 or an RNA encoding TGFbr3 in said first isolated population HSCs is less than or equal to a value representing a ratio of the amount of the housekeeping enzyme (e.g., GAPDH, actin beta or ubiquitin) or an RNA encoding said housekeeping enzyme and the amount of TGFbr3 or an RNA encoding TGFbr3 in said second isolated population HSCs when said second subject has fibrosis.

Preferably, in the methods described herein, the biological sample that is evaluated is liver tissue (e.g., a liver biopsy from a human) and the fibrosis that is screened for is liver fibrosis. In the methods described herein, the amount of TGFbr3 or an RNA encoding TGFbr3 and/or the amount of the housekeeping enzyme (e.g., GAPDH, actin or ubiquitin) or an RNA encoding said housekeeping enzyme are measured by QRTPCR.

More embodiments concern methods of identifying an agent that modulates the onset of fibrosis comprising providing a first population of HSCs; measuring the amount of TGFbr3 or an RNA encoding TGFbr3 in said first population of isolated HSCs; providing a second population of HSCs; providing a candidate agent; contacting said second population of isolated HSCs with said candidate agent; measuring the amount of TGFbr3 or an RNA encoding TGFbr3 in said second population of isolated HSCs after contact with said candidate agent; comparing the amount of TGFbr3 or an RNA encoding TGFbr3 measured in said first isolated population of HSCs to the amount of TGFbr3 or an RNA encoding TGFbr3 measured in said second isolated population of HSCs after contact with said candidate agent; and identifying the agent that modulates the onset of fibrosis when the amount of TGFbr3 or an RNA encoding TGFbr3 measured in said second isolated population of HSCs after contact with said candidate agent differs from the amount of TGFbr3 or an RNA encoding TGFbr3 measured in said first isolated population of HSCs. In some embodiments, the first and second isolated populations of HSCs are obtained from a subject (e.g., a human) that has fibrosis. In other embodiments, the first and second isolated populations of HSCs are obtained from a subject (e.g., a human) that does not have fibrosis.

These methods can be practiced such that the agent that inhibits the onset of fibrosis is identified when the amount of TGFbr3 or an RNA encoding TGFbr3 measured in said second isolated population of HSCs after contact with said candidate agent is greater than the amount of TGFbr3 or an RNA encoding TGFbr3 measured in said first isolated population of HSCs. These methods can also be practiced such that the agent that promotes the onset of fibrosis is identified when the amount of TGFbr3 or an RNA encoding TGFbr3 measured in said second isolated population of HSCs after contact with said candidate agent is less than the amount of TGFbr3 or an RNA encoding TGFbr3 measured in said first isolated population of HSCs.

In some embodiments, these methods further comprise measuring the amount of a housekeeping enzyme (e.g., GAPDH, actin, or ubiquitin) or an RNA encoding said housekeeping enzyme in said first and second isolated populations of HSCs. These methods can also further comprise combining a value representing the measured amount of TGFbr3 or an RNA encoding TGFbr3 in said first isolated population of HSCs and a value representing the measured amount of a housekeeping enzyme (e.g., GAPDH, actin, or ubiquitin) or an RNA encoding said housekeeping enzyme in said first isolated population of HSCs to obtain a first fibrosis score and comparing this first fibrosis score to a second fibrosis score obtained by combining a value representing the measured amount of TGFbr3 or an RNA encoding TGFbr3 in said second isolated population of HSCs after contact with said candidate agent and a value representing the measured amount of a housekeeping enzyme (e.g., GAPDH, actin, or ubiquitin) or an RNA encoding said housekeeping enzyme in said second isolated population of HSCs after contact with said candidate agent.

In some embodiments, the agent that modulates the onset of fibrosis is identified when the fibrosis score of said second population of HSCs is different than the fibrosis score of said first isolated population of HSCs after contact with said candidate agent. In other embodiments, the agent that inhibits the onset of fibrosis is identified when the fibrosis score of said second isolated population of HSCs after contact with said candidate agent is greater than the fibrosis score of said first isolated population of HSCs. In more embodiments, agent that promotes the onset of fibrosis is identified when the fibrosis score of said second isolated population of HSCs after contact with said candidate agent is less than the fibrosis score of said first isolated population of HSCs.

In still more embodiments, the methods above further comprise determining a ratio of the amount of a housekeeping enzyme (e.g., GAPDH, actin, or ubiquitin) or an RNA encoding said housekeeping enzyme and the amount of TGFbr3 or an RNA encoding TGFbr3 in said first isolated population of HSCs and comparing this ratio to a ratio of the amount a housekeeping enzyme (e.g., GAPDH, actin, or ubiquitin) or an RNA encoding said housekeeping enzyme and the amount of TGFbr3 or an RNA encoding TGFbr3 in said second isolated population of HSCs after contact with said candidate agent. In some embodiments, the agent that inhibits the onset of fibrosis is identified when the ratio of the amount of the housekeeping enzyme (e.g., GAPDH, actin, or ubiquitin) or an RNA encoding said housekeeping enzyme and the amount of TGFbr3 or an RNA encoding TGFbr3 in said first isolated population of HSCs is greater than the ratio of the amount of the housekeeping enzyme (e.g., GAPDH, actin, or ubiquitin) or an RNA encoding said housekeeping enzyme and the amount of TGFbr3 or an RNA encoding TGFbr3 in said second isolated population of HSCs after contact with said candidate agent. In more embodiments, the agent that promotes the onset of fibrosis is identified when the ratio of the amount of the housekeeping enzyme (e.g., GAPDH, actin, or ubiquitin) or an RNA encoding said housekeeping enzyme and the amount of TGFbr3 or an RNA encoding TGFbr3 in said first isolated population of HSCs is less than the ratio of the amount of the housekeeping enzyme (e.g., GAPDH, actin, or ubiquitin) or an RNA encoding said housekeeping enzyme and the amount of TGFbr3 or an RNA encoding TGFbr3 in said second isolated population of HSCs after contact with said candidate agent. Preferably, in these methods, first and second populations of HSCs are obtained from liver tissue (e.g., a liver biopsy from a human) and the fibrosis that is screened for is liver fibrosis. In these methods, the amount of TGFbr3 or an RNA encoding TGFbr3 and/or the amount of the housekeeping enzyme (e.g., GAPDH, actin, or ubiquitin) or an RNA encoding said housekeeping enzyme are measured by quantitative polymerase chain reaction. In some embodiments, the candidate agent is a retinoid compound, such as retinoic acid or a derivative or analog thereof.

More embodiments concern a method of making a formulation that inhibits the onset or progression of liver fibrosis in a subject (e.g., a human) comprising providing a first population of HSCs; measuring the amount of TGFbr3 or an RNA encoding TGFbr3 in said first population of isolated HSCs; providing a second population of HSCs; providing a candidate agent; contacting said second population of isolated HSCs with said candidate agent; measuring the amount of TGFbr3 or an RNA encoding TGFbr3 in said second population of isolated HSCs after contact with said candidate agent; comparing the amount of TGFbr3 or an RNA encoding TGFbr3 measured in said first isolated population of HSCs to the amount of TGFbr3 or an RNA encoding TGFbr3 measured in said second isolated population of HSCs after contact with said candidate agent; selecting an agent that increases the amount of TGFbr3 or an RNA encoding TGFbr3 in said second isolated population of HSCs; and formulating said agent for administration to a subject in need of an inhibition of the onset or progression of liver fibrosis.

Still more embodiments concern a method of inhibiting the onset or progression of liver fibrosis in a subject (e.g., a human) comprising providing a first population of HSCs; measuring the amount of TGFbr3 or an RNA encoding TGFbr3 in said first population of isolated HSCs; providing a second population of HSCs; providing a candidate agent; contacting said second population of isolated HSCs with said candidate agent; measuring the amount of TGFbr3 or an RNA encoding TGFbr3 in said second population of isolated HSCs after contact with said candidate agent; comparing the amount of TGFbr3 or an RNA encoding TGFbr3 measured in said first isolated population of HSCs to the amount of TGFbr3 or an RNA encoding TGFbr3 measured in said second isolated population of HSCs after contact with said candidate agent; selecting an agent that increases the amount of TGFbr3 or an RNA encoding TGFbr3 in said second isolated population of HSCs; providing said selected agent to a subject; and measuring an inhibition of the onset or progression of liver fibrosis in said subject.

Additional embodiments concern a method of identifying a predisposition for fibrosis in a tested subject (e.g., a human) comprising providing a biological sample that comprises a population of HSCs obtained from said tested subject; isolating said population of HSCs from said biological sample; measuring the amount of TGFbr3 or an RNA encoding TGFbr3 in said isolated population of HSCs; and identifying a predisposition for fibrosis in said tested subject when the amount of TGFbr3 or an RNA encoding TGFbr3 measured in said isolated population of HSCs is less than the standard amount of TGFbr3 or an RNA encoding TGFbr3 in HSCs isolated from normal liver or less than or equal to the standard amount of TGFbr3 or an RNA encoding TGFbr3 in HSCs isolated from fibrotic liver.

Methods of generating a fibrosis ratio are also embodiments and such methods are practiced by providing a biological sample comprising a population of HSCs; isolating said population of HSCs from said biological sample; measuring the amount of TGFbr3 or an RNA encoding TGFbr3 and measuring the amount of a housekeeping enzyme (e.g., GAPDH, actin, or ubiquitin) or an RNA encoding said housekeeping enzyme in said isolated population of HSCs; and generating a fibrosis ratio that represents the amount of TGFbr3 or an RNA encoding TGFbr3 in relation to the amount of said housekeeping enzyme (e.g., GAPDH, actin, or ubiquitin) or an RNA encoding said housekeeping enzyme in said isolated population of HSCs.

Methods of identifying a predisposition for fibrosis in a tested subject can also be practiced by providing a biological sample comprising a population of HSCs obtained from said tested subject (e.g., a human); isolating said population of HSCs from said biological sample; measuring the amount of TGFbr3 or an RNA encoding TGFbr3 and measuring the amount of a housekeeping enzyme (e.g., GAPDH, actin, or ubiquitin) or an RNA encoding said housekeeping enzyme in said isolated population of HSCs; generating a test subject fibrosis ratio that represents the amount of TGFbr3 or an RNA encoding TGFbr3 in relation to the amount of said housekeeping enzyme (e.g., GAPDH, actin, or ubiquitin) or an RNA encoding said housekeeping enzyme measured; comparing said test subject fibrosis ratio to a standard normal fibrosis ratio that represents the amount of TGFbr3 or an RNA encoding TGFbr3 in relation to the amount of said housekeeping enzyme (e.g., GAPDH, actin, or ubiquitin) or an RNA encoding said housekeeping enzyme in HSCs from a normal liver; or comparing said test subject fibrosis ratio to a standard abnormal fibrosis ratio that represents the amount of TGFbr3 or an RNA encoding TGFbr3 in relation to the amount of said housekeeping enzyme (e.g., GAPDH, actin, or ubiquitin) or an RNA encoding said housekeeping enzyme in HSCs from a fibrotic liver; and identifying a predisposition for fibrosis in said tested subject when the tested fibrosis ratio is less than the standard normal fibrosis ratio or less than or equal to the standard abnormal fibrosis ratio.

Additionally, methods of monitoring the progression of liver fibrosis or monitoring the efficacy of a treatment for liver fibrosis in a tested subject (e.g., a human) are embodiments and such methods can be practiced by providing a first biological sample comprising a population of HSCs obtained from said tested subject (e.g., a human) at a first time point; isolating said population of HSCs from said first biological sample; measuring the amount of TGFbr3 or an RNA encoding TGFbr3 and measuring the amount of a housekeeping enzyme (e.g., GAPDH, actin, or ubiquitin) or an RNA encoding said housekeeping enzyme in said isolated population of HSCs from said tested subject at said first time point; generating a first time point fibrosis ratio that represents the amount of TGFbr3 or an RNA encoding TGFbr3 in relation to the amount of said housekeeping enzyme (e.g., GAPDH, actin, or ubiquitin) or an RNA encoding said housekeeping enzyme measured at said first time point; providing a second biological sample comprising a population HSCs obtained from said tested subject at a second time point; isolating said population of HSCs from said second biological sample; measuring the amount of TGFbr3 or an RNA encoding TGFbr3 and measuring the amount of a housekeeping enzyme (e.g., GAPDH, actin, or ubiquitin) or an RNA encoding said housekeeping enzyme in said isolated population of HSCs from said tested subject at said second time point; generating a second time point fibrosis ratio that represents the amount of TGFbr3 or an RNA encoding TGFbr3 in relation to the amount of said housekeeping enzyme (e.g., GAPDH, actin, or ubiquitin) or an RNA encoding said housekeeping enzyme measured at said second time point; and comparing said first and said second fibrosis ratios to determine the progression of liver fibrosis or efficacy of said treatment for liver fibrosis.

Methods of identifying an agent that modulates the amount of TGFbr3 or an RNA encoding TGFbr3 in HSCs can also be practiced by providing a plurality of cells comprising HSCs; isolating a population of HSCs from said plurality of cells; contacting said population of HSCs or said plurality of cells with a candidate agent; measuring the amount of TGFbr3 and a housekeeping enzyme (e.g., GAPDH, actin, or ubiquitin) or an RNA encoding said housekeeping enzyme in said isolated population of HSCs; generating a candidate agent fibrosis ratio that represents the amount of TGFbr3 or an RNA encoding TGFbr3 in relation to the amount of said housekeeping enzyme (e.g., GAPDH, actin, or ubiquitin) or an RNA encoding said housekeeping enzyme measured; comparing said candidate agent fibrosis ratio to a standard normal fibrosis ratio that represents the amount of TGFbr3 or an RNA encoding TGFbr3 in relation to the amount of said housekeeping enzyme (e.g., GAPDH, actin, or ubiquitin) or an RNA encoding said housekeeping enzyme in HSCs from a normal liver when said plurality of cells comprising HSCs are obtained from normal liver; or comparing said candidate subject fibrosis ratio to a standard abnormal fibrosis ratio that represents the amount of TGFbr3 or an RNA encoding TGFbr3 in relation to the amount of said housekeeping enzyme (e.g., GAPDH, actin, or ubiquitin) or an RNA encoding said housekeeping enzyme in HSCs from a fibrotic liver when said plurality of cells comprising HSCs are obtained from fibrotic liver; and identifying said agent that modulates the amount of TGFbr3 or an RNA encoding TGFbr3 in HSCs when the candidate agent fibrosis ratio differs from the standard normal fibrosis ratio when said plurality of cells comprising HSCs are obtained from normal liver or when the candidate agent fibrosis ratio differs from the abnormal fibrosis ratio when said plurality of cells comprising HSCs are obtained from fibrotic liver.

### BRIEF DESCRIPTION OF THE DRAWINGS

FIG. 1 TGFbr3 expression of hepatic stellate cells isolated from normal (control or non-fibrotic) rat livers and hepatic stellate cells isolated from dimethyl nitrosamine (DMN)-treated rat livers, as measured by fluorescence activity after whole-transcript microarray analysis. Samples wt1-wt3 are the hepatic stellate cells (quiescent) isolated from the normal (untreated control or non-fibrotic) livers of three different rats. Samples fb4-fb6 are activated hepatic stellate cells (treated or experimental) isolated from the fibrotic livers of three different DMN-treated rats.

FIG. 2 Actin beta expression of hepatic stellate cells isolated from normal (control or non-fibrotic) rat livers and hepatic stellate cells isolated from DMN-treated rat livers, as measured by fluorescence activity after whole-transcript microarray analysis. Samples wt1-wt3 are the hepatic stellate cells (quiescent) isolated from the normal (untreated control or non-fibrotic) livers of three different rats. Samples fb4-fb6 are activated hepatic stellate cells (treated or experimental) isolated from the fibrotic livers of three different DMN-treated rats.

FIG. 3 GAPDH expression of hepatic stellate cells isolated from normal (control or non-fibrotic) rat livers and hepatic stellate cells isolated from DMN-treated rat livers, as measured by fluorescence activity after whole-transcript microarray analysis. Samples wt1-wt3 are the hepatic stellate cells (quiescent) isolated from the normal (untreated control or non-fibrotic) livers of three different rats. Samples fb4-fb6 are activated hepatic stellate cells (treated or experimental) isolated from the fibrotic livers of three different DMN-treated rats.

FIG. 4 GAPDH expression of hepatic stellate cells isolated from normal (control or non-fibrotic) rat livers and hepatic stellate cells isolated from DMN-treated rat livers, as measured by microarray. Wild type samples 1, 2 and 3 are the hepatic stellate cells (quiescent) isolated from normal (untreated control or non-fibrotic) livers of rats. Fibrosis samples 1, 2, and 3 are activated hepatic stellate cells (treated or experimental) isolated from the fibrotic livers of rats.

FIG. 5 TGFbr3 expression of hepatic stellate cells isolated from normal (control or non-fibrotic) rat livers and hepatic stellate cells isolated from DMN-treated rat livers, as measured by QRTPCR. Samples 1 and 2 are the hepatic stellate cells (quiescent) isolated from the normal (untreated control or non-fibrotic) livers of two different rats. Samples 3 and 4 are activated hepatic stellate cells (treated or experimental) isolated from the fibrotic livers of two different DMN-treated rats.

### DETAILED DESCRIPTION OF THE INVENTION

Described herein is the discovery that the onset of fibrosis is associated with a reduction in the amount of TGFbr3 or the level of expression of RNA encoding TGFbr3 in isolated HSCs. Microarray analysis conducted on HSCs that were isolated from the liver of animals with experimentally induced fibrosis revealed that these “activated” HSCs had a reduced amount of an RNA encoding TGFbr3, as compared to the amount of RNA encoding TGFbr3 found in “non-activated” HSCs isolated from normal (control) livers. QRTPCR conducted on HSCs isolated from fibrotic livers confirmed that the level or amount of RNA encoding TGFbr3 expression in HSCs that were isolated from fibrotic livers was significantly reduced compared to the amount or level of RNA encoding TGFbr3 in HSCs isolated from non-fibrotic or normal livers. Accordingly, a marker for the detection of fibrosis (e.g., liver fibrosis), for the identification of a predilection to acquire fibrosis (e.g., liver fibrosis), or to monitor the progression of fibrosis (e.g., liver fibrosis) was discovered.

It was also observed that as the level of fibrosis of the liver increased, the expression level of an RNA encoding TGFbr3 comparatively decreased. By normalizing the TGFbr3 expression levels with the expression levels of an RNA encoding a housekeeping enzyme (e.g., actin beta or GAPDH), a fibrosis ratio (a ratio of the amount of TGFbr3 or the amount of an RNA encoding TGFbr3 to the amount of the housekeeping enzyme or an RNA encoding a housekeeping enzyme) was generated. These fibrosis ratios can be used to diagnose or predict the stage of fibrosis, the predilection for having fibrosis, as well as, the progression of fibrosis or the efficacy of a treatment for fibrosis, for example, by comparing the fibrosis ratio of a particular sample to a standard curve or list of fibrosis ratios (e.g., a table of values or entries in literature) generated from analysis of normal and/or fibrotic tissue (e.g., fibrotic liver tissue).

Accordingly, methods of identifying a predisposition for fibrosis in a subject (e.g., a human) are described herein. By some approaches, a biological sample comprising a population of HSCs is obtained, the HSCs are isolated from the biological sample, and the amount of TGFbr3 or an RNA encoding TGFbr3 is measured or analyzed. The predisposition for fibrosis can then be identified or determined when the HSCs obtained from the biological sample show the presence of an amount of TGFbr3 or an RNA encoding TGFbr3 that is commensurate with the onset of fibrosis (e.g., a level of TGFbr3 protein or encoding RNA that is less than the level or amount found in normal HSCs or HSCs isolated from non-fibrotic tissue (e.g., non-fibrotic liver). By some approaches, the amount of TGFbr3 or an RNA encoding TGFbr3 that is measured in a first isolated population of HSCs obtained from a tested subject is compared to the amount of TGFbr3 or an RNA encoding TGFbr3 measured in a second isolated population of HSCs obtained from a subject that does not have fibrosis. In other embodiments, the amount of TGFbr3 or an RNA encoding TGFbr3 measured in a first isolated population of HSCs obtained from a tested subject is compared to the amount of TGFbr3 or an RNA encoding TGFbr3 measured in a second isolated population of HSCs obtained from a subject that has fibrosis. The predisposition for fibrosis can be identified when the amount of TGFbr3 or an RNA encoding TGFbr3 measured in said first isolated population of HSCs is less than the amount of TGFbr3 or an RNA encoding TGFbr3 measured in the second isolated population of HSCs obtained from said second biological sample from said second subject when said second subject does not have fibrosis; or when the amount of TGFbr3 or an RNA encoding TGFbr3 measured in said first isolated population of HSCs is less than or equal to the amount of TGFbr3 or an RNA encoding TGFbr3 measured in the second isolated population of HSCs obtained from said second biological sample from said second subject when said second subject has fibrosis.

As used herein, the term “isolated” requires that the material be removed from its original environment (e.g., the natural environment if it is naturally occurring). The term “enriched” means that the concentration of the material is at least about 2, 5, 10, 100, or 1000 times greater concentration than is found naturally occurring. The term “purified” does not require absolute purity; rather, it is intended as a relative definition. Purification, isolation or enrichment of HSCs to at least one order of magnitude, preferably two or three orders, and more preferably four or five orders of magnitude is expressly contemplated.

As described above, another aspect of the invention concerns methods of generating a fibrosis ratio, wherein a biological sample comprising HSCs is provided; a population of HSCs is isolated from the biological sample and the amount of TGFbr3 or an RNA encoding TGFbr3 and the amount of a housekeeping enzyme (e.g., actin, GAPDH, or ubiquitin) or an RNA encoding a housekeeping enzyme are measured. The fibrosis ratio is then generated by comparing or relating the amount of TGFbr3 or an RNA encoding TGFbr3 to the amount of the housekeeping enzyme or the RNA encoding the housekeeping enzyme. In some embodiments, a fibrosis score is developed by combining the value representing the amount of TGFbr3 or an RNA encoding TGFbr3 detected and the value representing the amount of the housekeeping enzyme or the RNA encoding the housekeeping enzyme detected. These fibrosis scores and fibrosis ratios can be compared to established parameters or ranges that represent fibrosis scores or fibrosis ratios that are predictive of the state of normal tissue (e.g., non-fibrosis tissue) and/or tissues that are abnormal (e.g., fibrotic tissue) so as to identify a predisposition for fibrosis and/or the efficacy of a particular treatment for fibrosis or a disease associated with fibrosis (e.g., hepatitis or alcohol dependence).

Similarly, methods of monitoring the progression of fibrosis (e.g., liver fibrosis) or monitoring the efficacy of a treatment for fibrosis (e.g., liver fibrosis) are contemplated. As above, a biological sample comprising HSCs from a tested subject (e.g., a human) is provided and said HSCs are isolated therefrom. The amount of TGFbr3 or an RNA encoding TGFbr3 is measured at a first time point (e.g., prior to treatment). At a second time point, a second biological sample comprising HSCs is obtained and a second population of HSCs is isolated from the second biological sample. The amount of TGFbr3 or an RNA encoding TGFbr3 in the HSCs isolated from the second biological sample at the second time point is then measured. By comparing the amount of TGFbr3 or an RNA encoding TGFbr3 measured at the first time point and the second time point one can readily determine the progression of fibrosis and/or the efficacy of the treatment for fibrosis. Optionally, the amount of a housekeeping enzyme (e.g., actin, GAPDH, or ubiquitin) or an RNA encoding a housekeeping enzyme (e.g., actin, GAPDH, or ubiquitin) is measured in said HSCs isolated from said first and second biological samples at said first and second time points and fibrosis scores or fibrosis ratios are generated. These fibrosis scores or fibrosis ratios can then be compared to a standard curve or list that indicates fibrosis scores or fibrosis ratios that are indicative of a normal or non-fibrotic tissue (e.g., liver) or fibrosis scores or fibrosis ratios that are indicative of abnormal or fibrotic tissue (e.g., liver). Preferably, the approaches described herein are applied to the analysis of the presence, level, or amount of TGFbr3 or an RNA encoding TGFbr3 in HSCs isolated from liver tissue and techniques including, but not limited to, microarray analysis, antibody detection, QRTPCR, test strips, western blot, sandwich assays, are used to analyze the presence, level, or amount of TGFbr3 or an RNA encoding TGFbr3 in the isolated HSCs.

Additional embodiments concern methods of identifying an agent or compound or mixture of agents or compounds that modulate (e.g., improve or inhibit) the amount of TGFbr3 or an RNA encoding TGFbr3 in HSCs or that modulate (e.g., improve or inhibit) the onset or progression of fibrosis in a tissue (e.g., liver) in a subject (e.g., a human). By some approaches, a first population of cells comprising HSCs is provided and the amount of TGFbr3 or an RNA encoding TGFbr3 is measured in HSCs isolated therefrom. A second population of cells comprising HSCs is then provided and the second population of cells or HSCs isolated therefrom are contacted with a candidate agent. After the contact with the candidate agent, the amount of TGFbr3 or an RNA encoding TGFbr3 is measured in the HSCs isolated from the second population of cells (e.g., the HSCs are subsequently isolated from the population of cells when the population of cells are contacted with the candidate agent or the isolated HSCs that are contacted with the agent are analyzed without further enrichment). The amount of TGFbr3 or an RNA encoding TGFbr3 measured in a control population of isolated HSCs (e.g., the isolated HSCs that were not contacted with the candidate agent) is compared to the amount of TGFbr3 or an RNA encoding TGFbr3 in the second population of isolated HSCs that were contacted with the candidate agent. The agent that modulates the amount of TGFbr3 or an RNA encoding TGFbr3 in HSCs or that modulates the onset or progression of fibrosis is identified when the amount of TGFbr3 or an RNA encoding TGFbr3 measured in the second population of isolated HSCs is greater than or less than the amount of TGFbr3 or an RNA encoding TGFbr3 measured in the first population (control), which did not come in contact with the candidate agent.

Optionally, a housekeeping enzyme (e.g., actin, GAPDH, or ubiquitin) or an RNA encoding a housekeeping enzyme (e.g., actin, GAPDH, or ubiquitin) is measured in said HSCs isolated from said first and second populations of HSCs and fibrosis scores or fibrosis ratios are generated. These fibrosis scores or fibrosis ratios can then be compared to a standard curve or list that indicates fibrosis scores or fibrosis ratios that are indicative of a normal or non-fibrotic tissue (e.g., liver) or fibrosis scores or fibrosis ratios that are indicative of abnormal or fibrotic tissue (e.g., liver). Accordingly, not only can a modulation of TGFbr3 levels or amounts be detected but a modulation event that may have significant therapeutic effect can be identified.

By some approaches, for example, a method of identifying an agent that modulates the amount of TGFbr3 or an RNA encoding TGFbr3 in HSCs is provided, wherein a plurality of cells comprising HSCs are provided and the plurality of cells or HSCs isolated therefrom are contacted with a candidate agent. The amount of TGFbr3 or an RNA encoding TGFbr3 and the amount of a housekeeping enzyme (e.g., actin, GAPDH, or ubiquitin) or an RNA encoding a housekeeping enzyme (e.g., actin, GAPDH, or ubiquitin) are measured in HSCs that are isolated from the plurality of cells that were contacted with the candidate agent (e.g., the HSCs are subsequently isolated from the plurality of cells when the plurality of cells are contacted with the candidate agent or the isolated HSCs that are contacted with the candidate agent are analyzed without further enrichment). A candidate agent fibrosis ratio that represents the amount of TGFbr3 or an RNA encoding TGFbr3 measured in relation to the amount of a housekeeping enzyme (e.g., actin, GAPDH, or ubiquitin) or an RNA encoding a housekeeping enzyme (e.g., actin, GAPDH, or ubiquitin) measured is then generated. The candidate agent fibrosis ratio can then be compared to a standard normal fibrosis ratio that represents the amount of TGFbr3 or an RNA encoding TGFbr3 in relation to the amount of said housekeeping enzyme or an RNA encoding a housekeeping enzyme in HSCs from a normal tissue when said plurality of cells comprising HSCs are obtained from normal tissue or the candidate subject fibrosis ratio can be compared to a standard abnormal fibrosis ratio that represents the amount of TGFbr3 or an RNA encoding TGFbr3 in relation to the amount of said housekeeping enzyme or an RNA encoding a housekeeping enzyme in HSCs from a fibrotic tissue when said plurality of cells comprising HSCs are obtained from fibrotic tissue.

The agent that modulates the amount of TGFbr3 or an RNA encoding TGFbr3 in HSCs can then be identified when the candidate agent fibrosis ratio differs from the standard normal fibrosis ratio when said plurality of cells comprising HSCs are obtained from normal tissue or when the candidate agent fibrosis ratio differs from the abnormal fibrosis ratio when said plurality of cells comprising HSCs are obtained from fibrotic tissue. Preferably, the approaches described herein are applied to the analysis of the presence, level, or amount of TGFbr3 or an RNA encoding TGFbr3 in HSCs isolated from liver tissue and techniques including, but not limited to, microarray analysis, antibody detection, QRTPCR, test strips, western blot, sandwich assays, are used to analyze the presence, level, or amount of TGFbr3 or an RNA encoding TGFbr3 in the isolated HSCs.

Agents that are identified by the methods described herein can be formulated into pharmaceuticals and provided to individuals that have fibrosis (e.g., liver fibrosis) or subjects that are at risk of having fibrosis (e.g., liver fibrosis). By some approaches, subjects (e.g., humans) identified as having a predilection to acquire fibrosis (e.g., identified by one or more of the diagnostic approaches described above) are provided an agent that inhibits fibrosis that has been identified by one of the methods described herein and, which has been formulated for treatment of fibrosis in humans or animals. Optionally, after being provided with one or more of the agents that inhibit fibrosis, which have been identified by a method described herein, the progression of fibrosis and therapeutic efficacy of the agent is monitored. As above, a biological sample comprising HSCs from a tested subject (e.g., a human) is provided and said HSCs are isolated therefrom. The amount of TGFbr3 or an RNA encoding TGFbr3 is measured at a first time point (e.g., prior to treatment with the agent) and at a second time point, a second biological sample comprising HSCs is obtained and a second population of HSCs is isolated from the second biological sample. The amount of TGFbr3 or an RNA encoding TGFbr3 in the HSCs isolated from the second biological sample at the second time point is then measured. By comparing the amount of TGFbr3 or an RNA encoding TGFbr3 measured at the first time point and the second time point one can readily determine the progression of fibrosis and/or the efficacy of the treatment for fibrosis.

Optionally, a housekeeping enzyme (e.g., actin, GAPDH, or ubiquitin) or an RNA encoding a housekeeping enzyme (e.g., actin, GAPDH, or ubiquitin) is measured in said HSCs isolated from said first and second biological samples at said first and second time points and fibrosis scores or fibrosis ratios are generated. These fibrosis scores or fibrosis ratios can then be compared to a standard curve or list that indicates fibrosis scores or fibrosis ratios that are indicative of a normal or non-fibrotic tissue (e.g., liver) or fibrosis scores or fibrosis ratios that are indicative of abnormal or fibrotic tissue (e.g., liver). Techniques that are useful for these analyses include, but not limited to, microarray analysis, antibody detection, QRTPCR, test strips, western blot, sandwich assays, are used to analyze the presence, level, or amount of TGFbr3 or an RNA encoding TGFbr3 in the isolated HSCs. The following section describes in greater detail several approaches that can be used to identify a predisposition or predilection to acquire fibrosis based on evaluating the presence, level, or amount of TGFbr3 or an RNA encoding TGFbr3 in an isolated population of HSCs.

Diagnostic Embodiments

Several diagnostic and prognostic methods described herein can be used to detect the concentration and/or expression level of nucleic acids encoding TGFbr3 and the concentration and/or amount of TGFbr3 protein in isolated HSCs and thereby gain an understanding of the current state of fibrosis of a subject (e.g., a human), a predilection of a subject to acquire fibrosis, a progression of fibrosis, or the therapeutic efficacy of a treatment for fibrosis. Generally, the diagnostic methods described herein can be classified according to whether the approach detects the concentration or expression level of a TGFbr3 nucleic acid or a TGFbr3 protein in a biological sample (e.g., HSCs isolated from liver tissue). Accordingly, the concentration and expression level of TGFbr3 in said biological sample can be determined by monitoring the amount of RNA in the sample. The detection of an abnormal amount RNA encoding TGFbr3 in a sample (e.g., an amount that is less than the amount found in HSCs isolated from non-fibrotic tissue) indicates the existence or predilection to acquire fibrosis or a fibrosis-related disease. Similarly, the concentration or amount of TGFbr3 in a biological sample can be determined by monitoring the amount of TGFbr3 protein in the sample. The detection of an abnormal amount of TGFbr3 in a sample (e.g., an amount that is less than the amount found in HSCs isolated from non-fibrotic tissue) indicates the existence or predilection to acquire a TGFbr3-related disease.

To determine the presence of TGFbr3 or TGFbr3 in a subject, first a biological sample having HSCs is obtained. Several methods known to those in the art can be employed to obtain a biological sample having HSCs (e.g., tissue biopsy). Once a biological sample from a subject in need of testing is obtained, many different techniques can be used to detect the concentration and/or expression level of TGFbr3 or TGFbr3 including, but not limited to, QRTPCR, microarray analysis, antibody-based detection techniques (e.g., enzyme-linked immunosorbant assays (ELISA), sandwich assays, immunoprecipitation, Western blots and immunoblots), bacteriophage display techniques, hybridization techniques (e.g., Southern and Northern), and enzymatic digestion (e.g., RNAse protection) techniques. Some of these techniques can involve disposing the proteins and/or nucleic acids present in the biological sample on a support, and contacting the support with detection reagents such as antibodies to TGFbr3 or nucleic acid probes complementary to TGFbr3 mRNA. Desirably, the levels of expression, amount, or concentration of TGFbr3 or TGFbr3 or both in HSCs isolated from fibrotic tissue and HSCs isolated from healthy tissue are compared to the level detected in the subject tested. Such comparisons can be made by reference to charts, lists, or standard curves that contain or refelect values indicative of HSCs isolated from fibrotic tissue and/or healthy tissue. Accordingly, a relative level of certainty of diagnosis can be gained by plotting the measured value or a representative score therefore on such a standard curve or by comparing the measured value to other measured values directly or indirectly (e.g., by comparing fibrosis ratios or fibrosis scores, as discussed supra) that may appear on lists or tables or discussion in the literature. In similar fashion, the techniques described above can be used to detect or determine the presence or amount of a housekeeping enzyme (e.g., actin, GAPDH, or ubiquitin) or an RNA encoding a housekeeping enzyme (e.g., actin, GAPDH, or ubiquitin).

In preferred embodiments, a microarray analysis is conducted to analyze the amount or level of expression of RNAs encoding TGFbr3 in an analyzed sample. (See Example 2). In some embodiments, addressable nucleic acid arrays comprise a plurality of nucleic acid probes that complement TGFbr3. These probes are joined to a support in different known locations. The knowledge of the precise location of each nucleic acid probe makes these “addressable” arrays particularly useful in binding assays. For example, an addressable array can comprise a support having several regions to which are joined a plurality nucleic acid probes that complement TGFbr3. The nucleic acids from a preparation of several biological samples from a plurality of human subjects (e.g., HSCs isolated from liver tissue biopsy samples) are labeled by conventional approaches (e.g., radioactivity or fluorescence) and the labeled samples are applied to the array under conditions that permit hybridization. If a nucleic acid in the sample hybridizes to a probe on the array, then a signal will be detected at a position on the support that corresponds to the location of the hybrid. Since the identity of each labeled sample is known and the region of the support on which the labeled sample was applied is known, an identification of the presence, concentration, and/or expression level can be rapidly determined. That is, by employing labeled standards of a known concentration of a nucleic acid (e.g., RNA) encoding TGFbr3, an investigator can accurately determine the concentration of the detected RNA encoding TGFbr3 in a sample and from this information can assess the expression level of TGFbr3. Conventional methods in densitometry can also be used to more accurately determine the concentration or expression level of an RNA encoding TGFbr3. These approaches are easily automated using technology known to those of skill in the art of high throughput diagnostic analysis. In similar fashion, the techniques described above can be used to detect or determine the presence or amount of an RNA encoding a housekeeping enzyme (e.g., actin, GAPDH, or ubiquitin).

Additionally, an opposite approach to that presented above can be employed. RNA obtained from biological samples (e.g., HSCs isolated from liver tissue) can be disposed on a support so as to create an addressable array. Preferably, the samples are disposed on the support at known positions that do not overlap. The presence of RNA encoding TGFbr3 in each sample is determined by applying labeled nucleic acid probes that complement the RNA that encode TGFbr3 and detecting the presence of a signal at locations on the array that correspond to the positions at which the biological samples were disposed. Because the identity of the biological sample and its position on the array is known, an identification of the presence, concentration, and/or expression level of the RNA encoding TGFbr3 is rapidly determined. That is, by employing labeled standards of a known concentration of an RNA encoding TGFbr3, an investigator can accurately determine the concentration of a nucleic acid encoding TGFbr3 in a sample and from this information can assess the expression level of TGFbr3. Conventional methods in densitometry can also be used to more accurately determine the concentration or expression level of an RNA encoding TGFbr3. These approaches are also easily automated using technology known to those of skill in the art of high throughput diagnostic analysis. In similar fashion, the techniques described above can be used to detect or determine the presence or amount of an RNA encoding a housekeeping enzyme (e.g., actin, GAPDH, or ubiquitin).

Any addressable array technology known in the art can be employed with this aspect of the invention. One particular embodiment of polynucleotide arrays is known as Genechips®, and has been generally described in U.S. Pat. No. 5,143,854; PCT publications WO 90/15070 and 92/10092. These arrays are generally produced using mechanical synthesis methods or light directed synthesis methods, which incorporate a combination of photolithographic methods and solid phase oligonucleotide synthesis. (Fodor et al., Science, 251:767-777, (1991)). The immobilization of arrays of oligonucleotides on solid supports has been rendered possible by the development of a technology generally identified as “Very Large Scale Immobilized Polymer Synthesis” (VLSIPS) in which, typically, probes are immobilized in a high density array on a solid surface of a chip. Examples of VLSIPS technologies are provided in U.S. Pat. Nos. 5,143,854 and 5,412,087 and in PCT Publications WO 90/15070, WO 92/10092 and WO 95/11995, which describe methods for forming oligonucleotide arrays through techniques such as light-directed synthesis techniques. In designing strategies aimed at providing arrays of nucleotides immobilized on solid supports, further presentation strategies were developed to order and display the oligonucleotide arrays on the chips in an attempt to maximize hybridization patterns and diagnostic information. Examples of such presentation strategies are disclosed in PCT Publications WO 94/12305, WO 94/11530, WO 97/29212 and WO 97/31256.

A wide variety of labels and conjugation techniques are known by those skilled in the art and can be used in various nucleic acid assays. There are several ways to produce labeled nucleic acids for hybridization or PCR (Polymerase Chain Reaction) including, but not limited to, oligolabeling, nick translation, end-labeling, or PCR amplification using a labeled nucleotide. Alternatively, a nucleic acid encoding TGFbr3, or any portion of it, can be cloned into a vector for the production of an mRNA probe. Such vectors are known in the art, are commercially available, and can be used to synthesize RNA probes in vitro by addition of an appropriate RNA polymerase such as T7, T3 or SP6 and labeled nucleotides. A number of companies supply commercial kits and protocols for these procedures. Suitable reporter molecules or labels include those radionuclides, enzymes, fluorescent, chemiluminescent, or chromogenic agents, as well as, substrates, cofactors, inhibitors, magnetic particles and the like.

For diagnostic and prognostic purposes, nucleic acid probes having a sequence complementary to a nucleic acid encoding TGFbr3 or a portion thereof can be used to detect and quantitate gene expression in biological samples. Preferably, nucleic acid probes that are complementary to mRNA encoding TGFbr3 are used to screen for polynucleotides present in HSCs isolated from tissues (e.g., liver). RNA-detection-based diagnostic assays, such as Northern hybridization, Northern dot blots, and RNA in situ hybridization are particularly useful to distinguish between the absence or presence or amount of TGFbr3 in a sample and to monitor TGFbr3 levels during progression of fibrosis or after therapeutic intervention. These techniques can also be employed to detect or determine the presence or amount of an RNA encoding a housekeeping enzyme (e.g., actin, GAPDH, or ubiquitin).

Some of the embodiments described herein may utilize oligonucleotide sequences, antisense RNA and DNA molecules, and PNAs that complement TGFbr3 sequences or RNAs encoding housekeeping enzymes for the determination of TGFbr3 concentrations and expression levels of RNAs encoding housekeeping enzymes in HSCs of a subject by RNA-based detection techniques. The form of such qualitative and/or quantitative methods can include Northern analysis, dot blot or other membrane-based technologies; PCR technologies; dip stick, pin, chip, and ELISA technologies. All of these techniques are well known in the art and are the basis of many commercially available diagnostic kits.

In one aspect, RNA probes complementary to TGFbr3 mRNA or an RNA encoding a housekeeping enzyme are used in assays that detect the presence of fibrosis or a predilection to acquire fibrosis. Accordingly, the nucleotide sequence encoding TGFbr3 or a housekeeping enzyme or a fragment thereof is used to design suitable RNA probes. The RNA probes are labeled by methods known in the art and are added to a DNAse treated fluid or tissue sample from a subject under conditions suitable for the formation of hybridization complexes. Hybridization complexes are isolated or the sample is treated with an agent that removes unhybridized nucleic acids. After an incubation period, the sample is washed with a compatible fluid that optionally contains a dye (or other label requiring a developer) if the nucleotide has been labeled with an enzyme. After the compatible fluid is rinsed off, the dye is quantitated and compared with a standard. If the amount of dye in the sample is significantly reduced over that of a comparable control sample, the presence of reduced levels of RNA encoding TGFbr3 or the housekeeping enzyme or a portion thereof in the sample is detected, which indicates the presence of fibrosis or a predilection to acquire fibrosis.

Such assays can also be used to evaluate the efficacy of a particular therapeutic treatment regime in animal studies, in clinical trials, or in monitoring the treatment of an individual patient. In order to provide a basis for the diagnosis of disease, a normal or standard profile for TGFbr3 expression in isolated HSCs cells is desirably established. This can be accomplished by combining HSCs taken from tissues (e.g., liver biopsy) from healthy subjects with RNA probes encoding TGFbr3, or a portion thereof, under conditions suitable for hybridization. Standard hybridization can be quantified by comparing the values obtained for healthy subjects and subjects having fibrosis with a dilution series of TGFbr3 RNA run in the same experiment where a known amount of substantially purified TGFbr3 is used. Standard values obtained from samples from healthy and diseased subjects are then compared with values obtained from samples from the tested subjects. Deviation between standards and the values obtained for the subject tested establishes the presence or predilection for fibrosis.

Additionally, PCR methods that can be used to quantitate the concentration and expression level of a particular molecule include radiolabeling (Melby P. C. et al. J Immunol Methods 159:235-44 (1993)) or biotinylating nucleotides (Duplaa C. et al. Anal Biochem 212:229-236 (1993)), coamplification of a control nucleic acid, and standard curves onto which the experimental results are interpolated. Quantitation of multiple samples can be processed more rapidly by running the assay in an ELISA format where the oligomer of interest is presented in various dilutions and a spectrophotometric or colorimetric response gives rapid quantitation. A definitive diagnosis of this type can allow health professionals to create a disease state profile for a patient, begin aggressive treatment for fibrosis, and prevent further worsening of the condition. Similarly, further assays and reference to the changing disease state profile can help clinicians monitor the progress of a patient during treatment. That is, once a disease state is established, a therapeutic agent is administered and an initial disease state profile is generated. The assays above can be repeated on a regular basis to evaluate whether the values in the subject's disease state profile progresses toward or returns back to the initial disease state profile. Successive treatment profiles can be used to show the efficacy of treatment over a period of several days or several months.

As mentioned above, PCR technology can be used to identify and quantitate concentration and expression levels of TGFbr3 or an RNA encoding a housekeeping enzyme. For amplification of mRNAs, it is within the scope of the present invention to reverse transcribe mRNA into cDNA followed by PCR (RT-PCR); or, to use a single enzyme for both steps as described in U.S. Pat. No. 5,322,770, the disclosure of which is incorporated herein by reference in its entirety, or, to use Reverse Transcriptase Asymmetric Gap Ligase Chain Reaction (RT-AGLCR), as described by Marshall R. L. et al. (PCR Methods and Applications 4:80-84, 1994), the disclosure of which is incorporated herein by reference in its entirety. Example 3 describes a QRTPCR approach that was used to evaluate the expression levels of TGFbr3 in HSCs isolated from liver tissue obtained from subjects having fibrosis and normal (non-fibrotic) subjects.

A variety of PCR techniques are familiar to those skilled in the art. For a review of PCR technology, see Molecular Cloning to Genetic Engineering White, B. A. Ed. in Methods in Molecular Biology 67: Humana Press, Totowa (1997), the disclosure of which is incorporated herein by reference in its entirety and the publication entitled “PCR Methods and Applications” (1991, Cold Spring Harbor Laboratory Press), the disclosure of which is incorporated herein by reference in its entirety. In each of these PCR procedures, PCR primers on either side of the TGFbr3 sequence to be amplified are added to a suitably prepared nucleic acid sample along with dNTPs and a thermostable polymerase such as Taq polymerase, Pfu polymerase, or Vent polymerase. The nucleic acid in the sample is denatured and the PCR primers are specifically hybridized to complementary nucleic acid sequences in the sample. The hybridized primers are extended. Thereafter, another cycle of denaturation, hybridization, and extension is initiated. The cycles are repeated multiple times to produce an amplified fragment containing the nucleic acid sequence between the primer sites. PCR has further been described in several patents including U.S. Pat. Nos. 4,683,195, 4,683,202 and 4,965,188, the disclosure of which is incorporated herein by reference in its entirety.

The primers are selected to be substantially complementary to a portion of the sequence of TGFbr3 mRNA and/or a RNA encoding a housekeeping enzyme and a portion of the sequence that complements the sequence of TGFbr3 mRNA and/or the housekeeping enzyme, thereby allowing the sequences between the primers to be amplified. The length of the primers for use with this aspect of the present invention can be identical to most of the lengths of the nucleic acid encoding TGFbr3 and/or the housekeeping enzyme. That is, primer length can be less than or equal to 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 41, 42, 43, 44, 45, 46, 47, 48, 49, or 50 nucleotides. Preferably, however primers are 16, 17, 18, 19, 20, 21, 22, 23, 24, 25, 26, 27, 28, 29 and 30 nucleotides in length. Shorter primers tend to lack specificity for a target nucleic acid sequence and generally require cooler temperatures to form sufficiently stable hybrid complexes with the template. Longer primers are expensive to produce and can sometimes self-hybridize to form hairpin structures. The formation of stable hybrids depends on the melting temperature (Tm) of the DNA. The Tm depends on the length of the primer, the ionic strength of the solution and the G+C content. The higher the G+C content of the primer, the higher is the melting temperature because G:C pairs are held by three H bonds whereas A:T pairs have only two. The G+C content of the amplification primers of the present invention preferably ranges between 10 and 75%, more preferably between 35 and 60%, and most preferably between 40 and 55%. The appropriate length for primers under a particular set of assay conditions may be empirically determined by one of skill in the art.

The presence of TGFbr3 protein or a housekeeping enzyme can be detected by screening for the presence of the protein using conventional assays. For example, monoclonal antibodies immunoreactive with TGFbr3 or a housekeeping enzyme can be used to screen biological samples for the presence, concentration, and amount of TGFbr3 or a housekeeping enzyme and, thereby, provide diagnostic information about the state or progression of fibrosis. Such immunological assays can be done in many convenient formats.

In one embodiment, antibodies are used to immunoprecipitate TGFbr3 and/or a housekeeping enzyme from solution and, in another embodiment, antibodies are used to react with TGFbr3 and/or a housekeeping enzyme on Western or Immunoblots of a polyacrylamide gel. In desirable embodiments, antibodies are used to detect TGFbr3 and/or a housekeeping enzyme in paraffin or frozen sections, using immunocytochemical techniques. Favored embodiments for detecting TGFbr3 and/or a housekeeping enzyme include ELISA, radioimmunoassays (RIA), immunoradiometric assays (IRMA) and immunoenzymatic assays (IEMA), including sandwich assays using monoclonal and/or polyclonal antibodies. Exemplary sandwich assays are described by David et al., in U.S. Pat. Nos. 4,376,110 and 4,486,530, hereby incorporated by reference.

In preferred protein-based diagnostic embodiments, antibodies specific for TGFbr3 and/or a housekeeping enzyme are attached to a support in an ordered array wherein a plurality of antibodies are attached to distinct regions of the support that do not overlap with each other. As with the nucleic acid-based arrays, the protein-based arrays are ordered arrays that are designed to be “addressable” such that the distinct locations are recorded and can be accessed as part of an assay procedure.

In some embodiments, addressable antibody arrays comprise a plurality of antibodies that recognize TGFbr3 and/or a housekeeping enzyme. These probes are joined to a support in different known locations. The knowledge of the precise location of each probe makes these “addressable” arrays particularly useful in binding assays. For example, an addressable array can comprise a support having several regions to which are joined a plurality antibody probes that recognize TGFbr3 and/or a housekeeping enzyme. Proteins from a preparation of several biological samples (e.g., HSCs isolated from liver tissue) from a plurality of human subjects or a plurality of tissues or fluids from a single subject are labeled by conventional approaches (e.g., radioactivity, colorimetrically, or fluorescently) and the labeled samples are applied to the array under conditions that permit binding. If a protein in the sample binds to an antibody probe on the array, then a signal will be detected at a position on the support that corresponds to the location of the antibody-protein complex. Since the identity of each labeled sample is known and the region of the support on which the labeled sample was applied is known, an identification of the presence, concentration, and/or expression level is rapidly determined. That is, by employing labeled standards of a known concentration of TGFbr3 and/or a housekeeping enzyme, an investigator can accurately determine the protein concentration of TGFbr3 and/or a housekeeping enzyme in a sample and from this information can assess the expression level of TGFbr3 and/or a housekeeping enzyme. Conventional methods in densitometry can also be used to more accurately determine the concentration or expression level of TGFbr3 and/or a housekeeping enzyme. These approaches are easily automated using technology known to those of skill in the art of high throughput diagnostic analysis.

In another embodiment, an opposite approach to that presented above can be employed. Proteins present in biological samples (e.g., tissues or fluids from one or more subjects or one or more sources in a subject's body) can be disposed on a support so as to create an addressable array. Preferably, the protein samples are disposed on the support at known positions that do not overlap. The presence of a protein encoding TGFbr3 and/or a housekeeping enzyme in each sample is then determined by applying labeled antibody probes that recognize epitopes of TGFbr3 and/or a housekeeping enzyme and detecting a signal at locations on the array that correspond to the positions at which the biological samples were disposed. Because the identity of the biological sample and its position on the array is known, an identification of the presence, concentration, and/or expression level TGFbr3 and/or a housekeeping enzyme is rapidly determined. That is, by employing labeled standards of a known concentration of TGFbr3 and/or a housekeeping enzyme, an investigator can accurately determine the concentration of TGFbr3 and/or a housekeeping enzyme in a sample and from this information can assess the expression level of TGFbr3 and/or a housekeeping enzyme. Conventional methods in densitometry can also be used to more accurately determine the concentration or expression level of TGFbr3 and/or a housekeeping enzyme. These approaches are also easily automated using technology known to those of skill in the art of high throughput diagnostic analysis. As detailed above, any addressable array technology known in the art can be employed with this aspect of the invention and display the protein arrays on the chips in an attempt to maximize antibody binding patterns and diagnostic information.

As discussed above, the presence or detection of TGFbr3 can provide a diagnosis of a subject's state of fibrosis or predilection to acquire fibrosis and this information allows health professionals to create a disease state profile for a patient, begin aggressive treatment for the fibrosis, and prevent further worsening of the condition. Similarly, further assays and reference to the changing disease state profile can help clinicians monitor the progress of a patient during treatment. That is, once a disease state is established, a therapeutic agent is administered and an initial disease state profile is generated. The assays above can be repeated on a regular basis to evaluate whether the values in the subject's disease state profile progresses toward or returns back to the initial disease state profile. Successive treatment profiles can be used to show the efficacy of treatment over a period of several days or several months. The following section describes several approaches that can be used to identify an agent that modulates the presence, level, or amount of TGFbr3 or an RNA encoding TGFbr3 in HSCs.

Approaches to Identify Agents that Modulate Fibrosis

The assays above can be used to evaluate the levels or amount of TGFbr3 or an RNA encoding TGFbr3 in isolated HSCs, and said levels or amounts of TGFbr3 or an RNA encoding TGFbr3 have been found to be related to the state of fibrosis in animals. The aforementioned assays can be used in approaches to screen for agents (e.g., agents obtained from commercially available compound libraries, such as compounds of the retinoid family, for instance, retinoic acid) that modulate (upregulate or downregulate or inhibit or enhance) the amount of TGFbr3 and/or the amount or level of expression of an RNA encoding TGFbr3 in HSCs and, thus, fibrosis in animals, as well (See Example 4).

By some methods, primary cultures of HSCs are established from animal tissue sources (e.g., liver tissue isolated from normal animals and/or animals with experimentally induced liver fibrosis) and these cultures of HSCs are contacted with a candidate agent (e.g., agents obtained from commercially available compound libraries, such as compounds of the retinoid family, for instance, retinoic acid or a derivative or analog thereof) and the level or amount of TGFbr3 or an RNA encoding TGFbr3 is measured, as described above. Agents that modulate (increase or reduce) the amount of TGFbr3 and/or the amount or level of expression of an RNA encoding TGFbr3 in HSCs are identified by selecting agents that induce an increase or decrease in the amount of TGFbr3 and/or the amount or level of expression of an RNA encoding TGFbr3 in the primary culture of HSCs after contact with said agent. Agents identified by these approaches can be formulated for the treatment of various forms of fibrosis in humans and animals, as described in greater detail below.

Alternatively, immortalized HSC cell lines can be used to identify agents that increase or decrease the amount of TGFbr3 and/or the amount or level of expression of an RNA encoding TGFbr3 and, thus, fibrosis in animals. The LX-1 and LX-2 cell lines, for example, can be used to screen for agents that modulate the amount of TGFbr3 and/or the amount or level of expression of an RNA encoding TGFbr3 in HSCs. (See e.g., Xu et al., Gut 54(1): 142-151 (2005), hereby expressly incorporated by reference in its entirety). As above, the LX-1 or LX-2 cells are contacted with a candidate agent (e.g., agents obtained from commercially available compound libraries, such as compounds of the retinoid family, for instance, retinoic acid or a derivative or analog thereof) in culture and the level or amount of TGFbr3 or an RNA encoding TGFbr3 is measured. Agents that modulate (increase or reduce) the amount of TGFbr3 and/or the amount or level of expression of an RNA encoding TGFbr3 in HSCs are identified by selecting agents that induce an increase or decrease in the amount of TGFbr3 and/or the amount or level of expression of an RNA encoding TGFbr3 in the LX-1 or LX-2 cells after contact with said agent. Agents identified by these approaches can be formulated for the treatment of various forms of fibrosis in humans and animals, as described in greater detail below

By still another approach, animals with experimentally induced fibrosis (e.g., DMN-treated animals) or normal animals (e.g., animal without fibrosis) are provided a candidate agent (e.g., agents obtained from commercially available compound libraries, such as compounds of the retinoid family of compounds, for instance, retinoic acid or a derivative or analog thereof), the livers or other desired tissues from the treated animals are removed and the HSCs are isolated therefrom. The amount of TGFbr3 and/or the amount or level of expression of an RNA encoding TGFbr3 is then analyzed using one or more of the diagnostic approaches above. Agents that modulate (increase or reduce) the amount of TGFbr3 and/or the amount or level of expression of an RNA encoding TGFbr3 in HSCs are identified by selecting agents that induce an increase or decrease in the amount of TGFbr3 and/or the amount or level of expression of an RNA encoding TGFbr3 in the HSCs isolated from animals that were contacted with the agent. Agents identified by these approaches can be formulated for the treatment of various forms of fibrosis in humans and animals, as described in greater detail below. In some embodiments, it may be desirable to generate primary cultures or immortalized cultures of HSCs isolated from these animals.

Many of these assays can be automated (e.g., high throughput screening employing a plurality of isolated HSCs) so as to quickly identify candidate agents that modulate the amount of TGFbr3 and/or the amount or level of expression of an RNA encoding TGFbr3. By some approaches, immortalized HSCs or primary cultures of HSCs are plated in multi-well petri dishes and the level or amount of TGFbr3 or an RNA encoding TGFbr3 is measured in a representative number of cells so as to obtain a baseline value representing the amount of TGFbr3 or RNA encoding TGFbr3. These values can be normalized by generating fibrosis ratios and fibrosis scores, based on the amount or level of housekeeping enzymes (e.g., actin, GAPDH, or ubiquitin) or RNAs encoding housekeeping enzymes, as described above. Once a baseline for TGFbr3 or an RNA encoding TGFbr3 is obtained, the plurality of wells having HSCs plated therein are contacted with different candidate agents (e.g., agents obtained from commercially available compound libraries, such as compounds of the retinoid family, for instance, retinoic acid). After contact with the candidate agent, the amount of TGFbr3 and/or the amount or level of expression of an RNA encoding TGFbr3 is analyzed in the contacted cells and agents that modulate the amount of TGFbr3 and/or the amount or level of expression of an RNA encoding TGFbr3 are identified by selecting agents that induce an increase or decrease in the amount of TGFbr3 and/or the amount or level of expression of an RNA encoding TGFbr3 in the HSCs after contact with said agent. Desirably, the assays are set-up in automated fashion and are addressable such that the identity of each compound that is contacted with each well is known. After contact with the candidate agent, the amount of TGFbr3 and/or the amount or level of expression of an RNA encoding TGFbr3 is measured using one or more of the diagnostic approaches described above. Agents identified by these approaches can be formulated for the treatment of various forms of fibrosis in humans and animals, as described in greater detail in the following section.

Pharmaceuticals Comprising Agents that Modulate Fibrosis

The agents identified by the methods described herein can be employed in admixture with conventional excipients, e.g., pharmaceutically acceptable organic or inorganic carrier substances suitable for parenteral, enteral (e.g., oral) or topical application that do not deleteriously react with the pharmacologically active ingredients. Suitable pharmaceutically acceptable carriers include, but are not limited to, water, salt solutions, alcohols, gum arabic, vegetable oils, benzyl alcohols, polyetylene glycols, gelatine, carbohydrates such as lactose, amylose or starch, magnesium stearate, talc, silicic acid, viscous paraffin, perfume oil, fatty acid monoglycerides and diglycerides, pentaerythritol fatty acid esters, hydroxy methylcellulose, polyvinyl pyrrolidone, etc. Many more suitable vehicles are described in Remmington's Pharmaceutical Sciences, 15th Edition, Easton:Mack Publishing Company, pages 1405-1412 and 1461-1487 (1975) and The National Formulary XIV, 14th Edition, Washington, American Pharmaceutical Association (1975), herein incorporated by reference. The pharmaceutical preparations can be sterilized and if desired mixed with auxiliary agents, e.g., lubricants, preservatives, stabilizers, wetting agents, emulsifiers, salts for influencing osmotic pressure, buffers, coloring, flavoring and/or aromatic substances and the like that do not deleteriously react with the actives.

The effective dose and method of administration of a particular pharmaceutical formulation having an agent identified by a method described herein can vary based on the individual needs of the patient and the treatment or preventative measure sought. Therapeutic efficacy and toxicity of such compounds can be determined by standard pharmaceutical procedures in cell cultures or experimental animals, e.g., ED50 (the dose therapeutically effective in 50% of the population). The dosage of such active ingredients lies preferably within a range of circulating concentrations that include the ED50 with no toxicity. The dosage varies within this range depending upon type of nucleic acids, peptides, and T cells described herein, the dosage form employed, sensitivity of the organism, and the route of administration.

Normal dosage amounts of an agent identified by a method described herein can vary from approximately 1 to 100,000 micrograms, up to a total dose of about 10 grams, depending upon the route of administration. Desirable dosages include 250 μg, 500 μg, 1 mg, 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 1 g, 1.1 g, 1.2 g, 1.3 g, 1.4 g, 1.5 g, 1.6 g, 1.7 g, 1.8 g, 1.9 g, 2 g, 3 g, 4 g, 5, 6 g, 7 g, 8 g, 9 g, and 10 g. The exact dosage is chosen by the individual physician in view of the patient to be treated. Dosage and administration are adjusted to provide sufficient levels of the active moiety or to maintain the desired effect. Additional factors that can be taken into account include the severity of the disease, age of the organism, and weight or size of the organism; diet, time and frequency of administration, drug combination(s), reaction sensitivities, and tolerance/response to therapy.

Routes of administration of the active ingredients described herein include, but are not limited to, topical, transdermal, parenteral, gastrointestinal, transbronchial, and transalveolar. Transdermal administration is accomplished by application of a cream, rinse, gel, etc. capable of allowing the pharmacologically active compounds to penetrate the skin. Parenteral routes of administration include, but are not limited to, electrical or direct injection such as direct injection into a central venous line, intravenous, intramuscular, intraperitoneal, intradermal, or subcutaneous injection. Gastrointestinal routes of administration include, but are not limited to, ingestion and rectal. Transbronchial and transalveolar routes of administration include, but are not limited to, inhalation, either via the mouth or intranasally.

Compositions having the pharmacologically actives identified as described herein that are suitable for transdermal or topical administration include, but are not limited to, pharmaceutically acceptable suspensions, oils, creams, and ointments applied directly to the skin or incorporated into a protective carrier such as a transdermal device (“transdermal patch”). Examples of suitable creams, ointments, etc. can be found, for instance, in the Physician's Desk Reference.

Compositions having the pharmacologically actives identified as described herein that are suitable for parenteral administration include, but are not limited to, pharmaceutically acceptable sterile isotonic solutions. Such solutions include, but are not limited to, saline and phosphate buffered saline for injection into a central venous line, intravenous, intramuscular, intraperitoneal, intradermal, or subcutaneous injection.

Compositions having the pharmacologically actives identified as described herein that are suitable for transbronchial and transalveolar administration include, but not limited to, various types of aerosols for inhalation. Devices suitable for transbronchial and transalveolar administration of these are also embodiments. Such devices include, but are not limited to, atomizers and vaporizers. Many forms of currently available atomizers and vaporizers can be readily adapted to deliver compositions having the pharmacologically actives described herein.

Compositions having the pharmacologically actives identified as described herein that are suitable for gastrointestinal administration include, but not limited to, pharmaceutically acceptable powders, pills or liquids for ingestion and suppositories for rectal administration.

### Example 1

This Example describes in greater detail some of the approaches used to establish the animal model of fibrosis and the isolation of HSCs from normal and fibrotic liver tissue.

Animal Models of Liver Fibrosis

All animals were housed in a facility approved by the American Association for Accreditation of Laboratory Animal Care and received care in compliance with specified guidelines. Animals were maintained under standard conditions. Liver fibrosis was induced by DMN treatment. Briefly, male Sprague-Dawley rats, weighing about 500 g, were injected (ip) with 10 mg/kg DMN every other day for 30 days total. Rats were then anesthetized with Xylazin (2% Xylazinhydrochlorid, 0.7% Methyl-4-hydroxybenzoate), and were sacrificed after removal of their livers.

Isolation of HSCs

HSCs from normal and fibrotic livers were isolated using an enzymatic perfusion method and subsequent fractionation of the heterogenous cell suspension on a continuous density gradient composed of Nycodenz in GBSS/B. Briefly, dissected livers were perfused with GBSS/B, Pronase E, Collagenase D and Dnase I sequentially at speed of 12-15 ml/min perfusion for 10 minutes for each step. Then, the livers were minced and further digested in a solution containing 0.5 mg/ml of Pronase E, 0.5 mg/ml of Collagenase, and 16.7 ug/ml of Dnase I at 37° C. for 30 minutes. The dissaggregated liver cells were then filtered through a nylon filter having a 100 um pore size and the cells were collected by centrifugation. Accordingly, the liver cells were resuspended in 10 ml of GBSS/B containing 24 ug/ml of Dnase I and approximately, 1 ml of the cells were layered on 11.5 ml of the 27.5% of Nycodenz in GBSS/B gradient. The mixture was separated by centrifugation at 1500 g for 17 minutes at 4° C. and the HCSs were isolated by removing the band located between the upper (clear) GBSS/B solution and lower Nycodenz solution.

### Example 2

This example describes a first approach that was used to evaluate the expression levels of TGFbr3, actin beta, and GAPDH, in HSCs isolated from normal and fibrotic livers.

Microarray Analysis

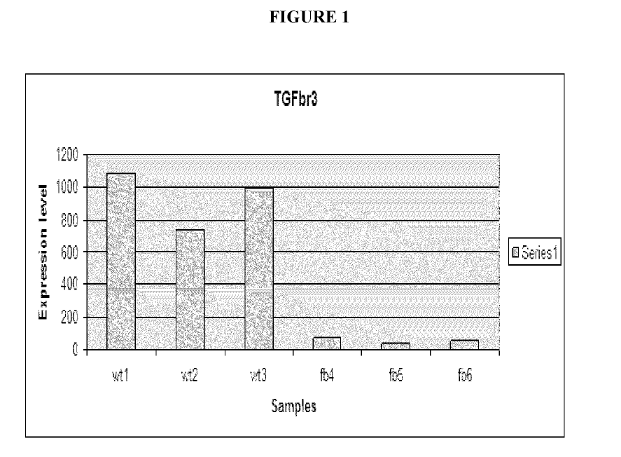

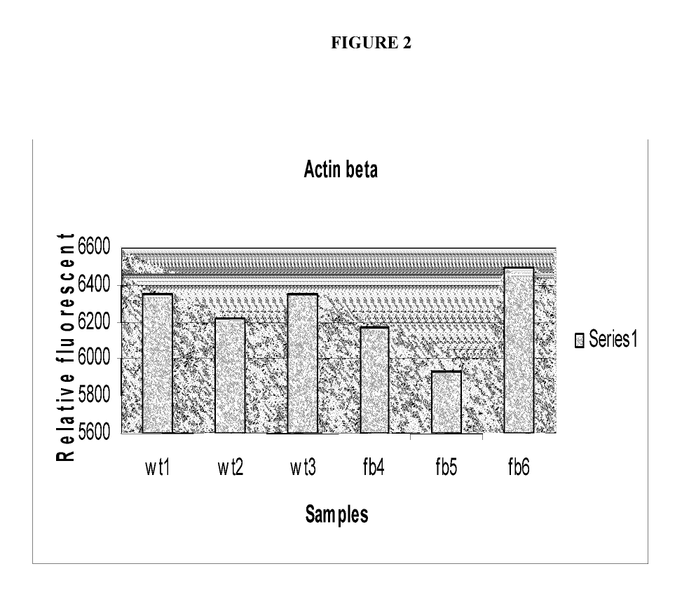

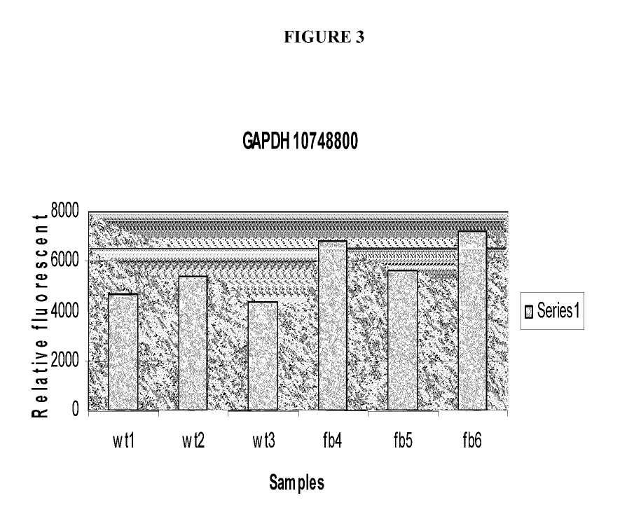

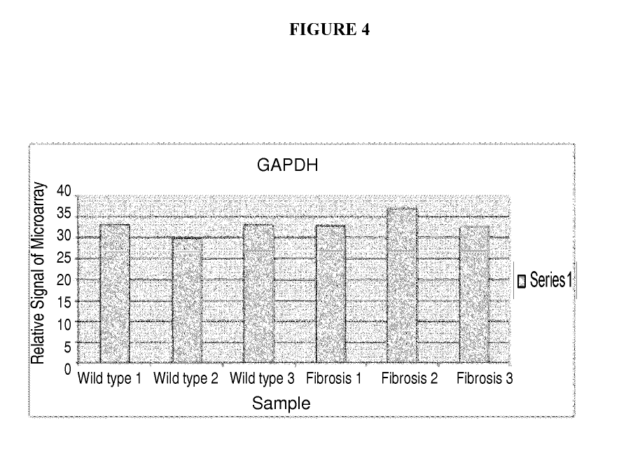

Microarray studies were carried out using the Affymetrix® whole-transcript microarray, GeneChip® Rat Gene 1.0 ST Array, which displays oligonucleotides corresponding to 27,342 genes of the rat genome (Affymetrix®, Santa Clara, Calif.). Triplicate RNA samples obtained from HSCs that were isolated from individual rat livers, which were either Normal (e.g., control rats that have not been contacted with DMN having non-fibrotic livers) or Abnormal (e.g., DMN-treated rats having fibrotic livers) were hybridized to single Affymetrix® microarray chips and the results were analyzed using the Affymetrix® Microarray GCOS software. GeneSpring® Software GX 7.0 (Silicon Genetics®, Redwood City, Calif.) was used for analyzing the differential expression and data annotation. Genes were defined as being differentially expressed if they were down-regulated in the HSCs obtained from DMN-treated rats, as compared to the level of expression seen in HSCs obtained from Normal rats, or if they were up-regulated in the HSCs obtained from DMN-treated rats, as compared to the level of expression seen in HSCs obtained from Normal rats. The desired selection criteria included a fold change that was greater than 0.01. The NetAffx® database (Affymetrix®, Santa Clara, Calif.) was used for determining the identity of differentially expressed genes. The gene expression results were also analyzed using GSEA (Gene-Set Enrichment Analysis) to identify global differences in pathways. The results shown in FIG. 1, reveal that the amount of TGFbr3 in the HSCs isolated from fibrotic animals is significantly reduced as compared to the amount of TGFbr3 in HSCs isolated from normal animals. The results shown in FIGS. 2-4 show that the amounts of house keeping enzymes in the HSCs isolated from normal animals and fibrotic animals are approximately the same. Based on this data, a fibrosis score and/or fibrosis ration can be developed.

### Example 3

This example describes a second approach that was used to evaluate the expression levels of TGFbr3 in HSCs isolated from normal and fibrotic livers.

Quantitative Reverse Transcriptase PCR (QRTPCR) Analysis

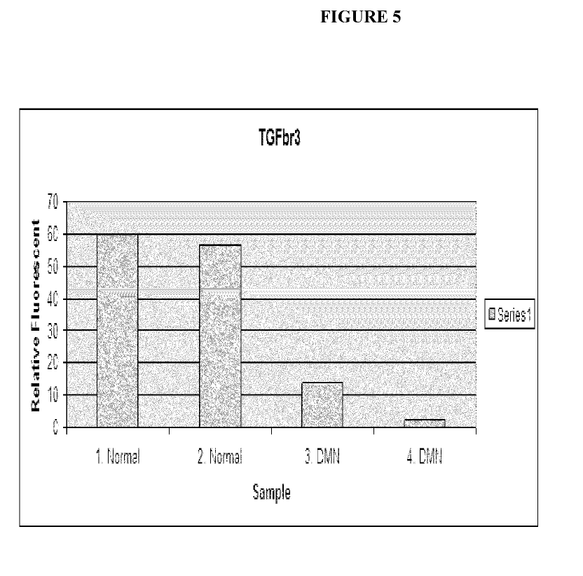

QRTPCR was also used to evaluate the expression levels of TGFbr3 and GAPDH, in HSCs isolated from normal and fibrotic livers. RNA was obtained from the HSCs that were isolated from the livers of Normal rats (e.g., control rats that have not been contacted with DMN having non-fibrotic livers) or Abnormal rats (e.g., DMN-treated rats having fibrotic livers). The QRTPCR results were normalized to the internal expression controls of GAPDH (glyceraldehyde-3-phosphate dehydrogenase). The QRTPCR primers were designed and prepared using a web-based software program and a commercially available master mix QRTPCR kit was used. All cDNA was prepared with Bio-Rad® cDNA synthesis kit according to manufacturer's instructions (Bio-Rae). A total of 25 ng of cDNA was used in each individual reaction in total 20 ul of QRTPCR reaction solution with 312 nmol of primers of each. The ABI® 7900HT Fast Real-Time PCR System (ABI) was used for detection and quantitation of PCR products. Time cycles were set for 5 minutes at 50° C., 15 minutes at 94° C. followed by 40 cycles of 15 seconds at 94° C., minutes at 60° C. Artificial standard curves were generated by the expression levels of glyceraldehyde-3-phosphate dehydrogenase using cDNA generated from 1 ng to 100 ng total RNA for relative quantitation. All sample signals were normalized to a signal corresponding to GAPDH from 25 ng of total RNA. The results shown in FIG. 5 again show that the amount of TGFbr3 in HSCs isolated from animals with fibrosis is significantly less than the amount of TGFbr3 in HSCs isolated from normal animals. Accordingly, a very sensitive assay to diagnose the onset and progression of fibrosis was discovered.

### Example 4

This example describes an approach to identify an agent that modulates the amount of TGFbr3 or an RNA encoding TGFbr3 in HSCs. In a first series of experiments, primary cultures of HSCs are established from the liver of rats. Desirably, in a first group, primary cultures of HSCs are established from normal rats (e.g., rats that have non-fibrotic livers) and, in a second group, primary cultures of HSCs are established from rats that have been treated with DMN so as to induce fibrosis (see e.g., Examples 1-3). Preferably, the HSCs are cultured in multi-well petri dishes (e.g., 64 well dishes) in suitable media. Some of the HSCs from the two groups of primary cultures are isolated and, prior to treatment with a candidate agent, the amount of an RNA that encodes TGFbr3 is determined using QRTPCR (see Example 3). Candidate agents (e.g., a plurality of different compounds in the retinoid family, such as retinoic acid and derivatives or analogs thereof) are contacted with the two groups of HSCs by applying each individual compound singularly to each well of the petri dish. Desirably, each compound is tested with HSCs from the first and second groups so as to evaluate the impact each compound has on activated and nonactivated HSCs. At different time points after the contact with the candidate agent, the HSCs from the first and second groups are isolated and the amount of an RNA that encodes TGFbr3 is measured using QRTPCR (see Example 3). Optionally, the amount of an RNA that encodes a housekeeping enzyme (e.g., actin beta or GAPDH) is measured before and after contact with the candidate agent. Desirably, fibrosis ratios are generated by relating the amount of RNA encoding TGFbr3 measured to the amount of RNA encoding a housekeeping enzyme. Agents that modulate the amount of TGFbr3 or an RNA encoding TGFbr3 in HSCs are identified by selecting the compounds that induced a change or a difference in the amount of an RNA encoding TGFbr3 or the fibrosis ratios of the HSCs after contact with the candidate agent. It is contemplated that compounds in the retinoid family will modulate the amount of an RNA encoding TGFbr3 in primary cultures of HSCs isolated from normal livers and/or fibrotic livers.

In a second series of experiments, immortalized cell lines LX-1 or LX-2 are used to identify agents that modulate the amount of TGFbr3 or an RNA encoding TGFbr3 in HSCs. Preferably, the HSCs are cultured in multi-well petri dishes (e.g., 64 well dishes) in suitable media (see e.g., Xu et al., Gut 54(1): 142-151 (2005). As above, preferably, a baseline of the amount of an RNA encoding TGFbr3 is established prior to contacting the HSCs with a candidate agent. The amount of an RNA encoding TGFbr3 is measured using QRTPCR (see Example 3) prior to contacting the LX-1 or LX-2 cells with a candidate agent. As above, candidate agents (e.g., a plurality of different compounds in the retinoid family, such as retinoic acid and derivatives and analogs thereof) are contacted with the immortalized HSCs by applying each individual compound singularly to each well of the petri dish. At different time points after the contact with the candidate agent, the HSCs are isolated and the amount of an RNA that encodes TGFbr3 is measured using QRTPCR (see Example 3). Optionally, the amount of an RNA that encodes a housekeeping enzyme (e.g., actin beta or GAPDH) is measured before and after contact with the candidate agent. Desirably, fibrosis ratios are generated by relating the amount of RNA encoding TGFbr3 measured to the amount of RNA encoding a housekeeping enzyme. Agents that modulate the amount of TGFbr3 or an RNA encoding TGFbr3 in the immortalized HSCs are identified by selecting the compounds that induced a change or a difference in the amount of an RNA encoding TGFbr3 or the fibrosis ratios of the HSCs after contact with the candidate agent. It is contemplated that compounds in the retinoid family will modulate the amount of an RNA encoding TGFbr3 in immortalized cultures of HSCs isolated from normal livers and/or fibrotic livers.

Although the invention has been described with reference to embodiments and examples, it should be understood that various modifications can be made without departing from the spirit of the invention. Accordingly, the invention is limited only by the following claims. All references cited herein are hereby expressly incorporated by reference.

What is claimed is:

1. A method of identifying a predisposition for fibrosis in a tested subject comprising:
providing a first biological sample comprising a first population of hepatic stellate cells (HSCs) obtained from said tested subject;
isolating said first population of HSCs from said first biological sample;
measuring the amount of Transforming Growth Factor beta receptor III (TGFbr3) or an RNA encoding TGFbr3 in said first isolated population of HSCs;
comparing the amount of TGFbr3 or an RNA encoding TGFbr3 measured in said first isolated population of HSCs to an amount of TGFbr3 or an RNA encoding TGFbr3 measured in a second population of isolated HSCs obtained from a second biological sample from a second subject; and
identifying a predisposition for fibrosis in said tested subject when:
(a) the amount of TGFbr3 or an RNA encoding TGFbr3 measured in said first isolated population of HSCs is less than the amount of TGFbr3 or an RNA encoding TGFbr3 measured in the second isolated population of HSCs obtained from said second biological sample from said second subject when said second subject does not have fibrosis; or
(b) the amount of TGFbr3 or an RNA encoding TGFbr3 measured in said first isolated population of HSCs is less than or equal to the amount of TGFbr3 or an RNA encoding TGFbr3 measured in the second isolated population of HSCs obtained from said second biological sample from said second subject when said second subject has fibrosis.

2. The method of claim 1, further comprising measuring the amount of a housekeeping enzyme (e.g., GAPDH, actin beta, or ubiquitin) or an RNA encoding a housekeeping enzyme (e.g., GAPDH, actin beta, or ubiquitin) in said first isolated population of HSCs and said second isolated population of HSCs.

3. The method of claim 2, further comprising combining a value representing the measured amount of TGFbr3 or an RNA encoding TGFbr3 in said first isolated population of HSCs and a value representing the measured amount of a housekeeping enzyme (e.g., GAPDH, actin beta, or ubiquitin) or an RNA encoding a housekeeping enzyme (e.g., GAPDH, actin beta, or ubiquitin) in said first isolated population of HSCs to obtain a first fibrosis score and comparing this first fibrosis score to a second fibrosis score obtained by combining a value representing the measured amount of TGFbr3 or an RNA encoding TGFbr3 in said second isolated population of HSCs and a value representing the measured amount of a housekeeping enzyme (e.g., GAPDH, actin beta, or ubiquitin) or an RNA encoding a housekeeping enzyme (e.g., GAPDH, actin beta, or ubiquitin) in said second isolated population of HSCs.

4. The method of claim 3, wherein the predisposition for fibrosis in said tested subject is identified when:
(a) the fibrosis score of the tested subject is less than the fibrosis score of said second subject when said second subject does not have fibrosis; and
(b) the fibrosis score of the tested subject is equal to, or less than the fibrosis score of said second subject when said second subject has fibrosis.

5. The method of claim 2, further comprising determining a ratio of the amount of a housekeeping enzyme (e.g., GAPDH, actin beta, or ubiquitin) or an RNA encoding a housekeeping enzyme (e.g., GAPDH, actin beta, or ubiquitin) and the amount of TGFbr3 or a RNA encoding TGFbr3 in said first population of isolated HSC and comparing this ratio to a ratio of the amount of a housekeeping enzyme (e.g., GAPDH, actin beta, or ubiquitin) or an RNA encoding a housekeeping enzyme (e.g., GAPDH, actin beta, or ubiquitin) and the amount of TGFbr3 or an RNA encoding TGFbr3 in said second isolated population of HSCs.

6. The method of claim 5, wherein the predisposition of fibrosis in said tested subject is identified when a value representing the ratio of the amount of a housekeeping enzyme (e.g., GAPDH, actin beta, or ubiquitin) or an RNA encoding a housekeeping enzyme (e.g., GAPDH, actin beta, or ubiquitin) and the amount of TGFbr3 or an RNA encoding TGFbr3 in said first isolated population HSCs is less than a value representing a ratio of the amount of a housekeeping enzyme (e.g., GAPDH, actin beta, or ubiquitin) or an RNA encoding a housekeeping enzyme (e.g., GAPDH, actin beta, or ubiquitin) and the amount of TGFbr3 or an RNA encoding TGFbr3 in said second isolated population HSCs when said second subject does not have fibrosis.

7. The method of claim 5, wherein the predisposition of fibrosis in said tested subject is identified when a value representing the ratio of the amount of a housekeeping enzyme (e.g., GAPDH, actin beta, or ubiquitin) or an RNA encoding a housekeeping enzyme (e.g., GAPDH, actin beta, or ubiquitin) and the amount of TGFbr3 or an RNA encoding TGFbr3 in said first isolated population HSCs is less than or equal to a value representing a ratio of the amount of a housekeeping enzyme (e.g., GAPDH, actin beta, or ubiquitin) or an RNA encoding a housekeeping enzyme (e.g., GAPDH, actin beta, or ubiquitin) and the amount of TGFbr3 or an RNA encoding TGFbr3 in said second isolated population HSCs when said second subject has fibrosis.

8. The method of claim 1, wherein said biological sample is liver tissue.

9. The method of claim 1, wherein said fibrosis is liver fibrosis.

10. The method of claim 1, wherein the amount of TGFbr3 is measured by quantitative reverse transcriptase polymerase chain reaction (QRTPCR).

11. The method of claim 2, wherein the amount of GAPDH, actin beta or ubiquitin is measured by QRTPCR.

12. A method of identifying an agent that modulates the onset of fibrosis comprising:
providing a first population of hepatic stellate cells (HSCs);
measuring the amount of Transforming Growth Factor beta receptor III (TGFbr3) or an RNA encoding TGFbr3 in said first population of isolated HSCs;
providing a second population of HSCs;
providing a candidate agent;
contacting said second population of isolated HSCs with said candidate agent;
measuring the amount of TGFbr3 or an RNA encoding TGFbr3 in said second population of isolated HSCs after contact with said candidate agent;
comparing the amount of TGFbr3 or an RNA encoding TGFbr3 measured in said first isolated population of HSCs to the amount of TGFbr3 or an RNA encoding TGFbr3 measured in said second isolated population of HSCs after contact with said candidate agent; and
identifying the agent that modulates the onset of fibrosis when the amount of TGFbr3 or an RNA encoding TGFbr3 measured in said second isolated population of HSCs after contact with said candidate agent differs from the amount of TGFbr3 or an RNA encoding TGFBr3 measured in said first isolated population of HSCs.

13. The method of claim 12, wherein the first and second isolated populations of HSCs are obtained from a subject that has fibrosis.

14. The method of claim 12, wherein the first and second isolated populations of HSCs are obtained from a subject that does not have fibrosis.

15. The method of claim 12, wherein an agent that inhibits the onset of fibrosis is identified when the amount of TGFbr3 or an RNA encoding TGFbr3 measured in said second isolated population of HSCs after contact with said candidate agent is greater than the amount of TGFbr3 or an RNA encoding TGFbr3 measured in said first isolated population of HSCs.

16. The method of claim 12, wherein an agent that promotes the onset of fibrosis is identified when the amount of TGFbr3 or an RNA encoding TGFbr3 measured in said second isolated population of HSCs after contact with said candidate agent is less than the amount of TGFbr3 or an RNA encoding TGFbr3 measured in said first isolated population of HSCs.

17. The method of claim 12, further comprising measuring the amount of a housekeeping enzyme (e.g., GAPDH, actin beta, or ubiquitin) or an RNA encoding a housekeeping enzyme (e.g., GAPDH, actin beta, or ubiquitin) in said first and second isolated populations of HSCs.

18. The method of claim 17, further comprising combining a value representing the measured amount of TGFbr3 or an RNA encoding TGFbr3 in said first isolated population of HSCs and a value representing the measured amount of a housekeeping enzyme (e.g., GAPDH, actin beta, or ubiquitin) or an RNA encoding a housekeeping enzyme (e.g., GAPDH, actin beta, or ubiquitin) in said first isolated population of HSCs to obtain a first fibrosis score and comparing this first fibrosis score to a second fibrosis score obtained by combining a value representing the measured amount of TGFbr3 or an RNA encoding TGFbr3 in said second isolated population of HSCs after contact with said candidate agent and a value representing the measured amount of a housekeeping enzyme (e.g., GAPDH, actin beta, or ubiquitin) or an RNA encoding a housekeeping enzyme (e.g., GAPDH, actin beta, or ubiquitin) in said second isolated population of HSCs after contact with said candidate agent.

19. The method of claim 18, wherein the agent that modulates the onset of fibrosis is identified when the fibrosis score of said second population of HSCs is different than the fibrosis score of said first isolated population of HSCs after contact with said candidate agent.

20. The method of claim 19, wherein an agent that inhibits the onset of fibrosis is identified when the fibrosis score of said second isolated population of HSCs after contact with said candidate agent is greater than the fibrosis score of said first isolated population of HSCs.

21. The method of claim 19, wherein an agent that promotes the onset of fibrosis is identified when the fibrosis score of said second isolated population of HSCs after contact with said candidate agent is less than the fibrosis score of said first isolated population of HSCs.

22. The method of claim 17, further comprising determining a ratio of the amount of a housekeeping enzyme (e.g., GAPDH, actin beta, or ubiquitin) or an RNA encoding a housekeeping enzyme (e.g., GAPDH, actin beta, or ubiquitin) and the amount of TGFbr3 or an RNA encoding TGFbr3 in said first isolated population of HSCs and comparing this ratio to a ratio of the amount of a housekeeping enzyme (e.g., GAPDH, actin beta, or ubiquitin) or an RNA encoding a housekeeping enzyme (e.g., GAPDH, actin beta, or ubiquitin) and the amount of TGFbr3 or a RNA encoding TGFbr3 in said second isolated population of HSCs after contact with said candidate agent.

23. The method of claim 22, wherein an agent that inhibits the onset of fibrosis is identified when the ratio of the amount of a housekeeping enzyme (e.g., GAPDH, actin beta, or ubiquitin) or an RNA encoding a housekeeping enzyme (e.g., GAPDH, actin beta, or ubiquitin) and the amount of TGFbr3 or an RNA encoding TGFbr3 in said first isolated population of HSCs is greater than the ratio of the amount of a housekeeping enzyme (e.g., GAPDH, actin beta, or ubiquitin) or an RNA encoding a housekeeping enzyme (e.g., GAPDH, actin beta, or ubiquitin) and the amount of TGFbr3 or an RNA encoding TGFbr3 in said second isolated population of HSCs after contact with said candidate agent.

24. The method of claim 22, wherein an agent that promotes the onset of fibrosis is identified when the ratio of the amount of a housekeeping enzyme (e.g., GAPDH, actin beta, or ubiquitin) or an RNA encoding a housekeeping enzyme (e.g., GAPDH, actin beta, or ubiquitin) and the amount of TGFbr3 or an RNA encoding TGFbr3 in said first isolated population of HSCs is less than the ratio of the amount of a housekeeping enzyme (e.g., GAPDH, actin beta, or ubiquitin) or an RNA encoding a housekeeping enzyme (e.g., GAPDH, actin beta, or ubiquitin) and the amount of TGFbr3 or an RNA encoding TGFbr3 in said second isolated population of HSCs after contact with said candidate agent.

25. The method of claim 12, wherein said first and second populations of HSCs are obtained from liver tissue.

26. The method of claim 12, wherein said fibrosis is liver fibrosis.

27. The method of claim 12, wherein the amount of TGFbr3 or an RNA encoding TGFbr3 is measured by QRTPCR.

28. The method of claim 17, wherein the amount of a housekeeping enzyme (e.g., GAPDH, actin beta, or ubiquitin) or an RNA encoding a housekeeping enzyme (e.g., GAPDH, actin beta, or ubiquitin) is measured by QRTPCR.

29. The method of claim 17, wherein the candidate agent is a retinoid compound.

30. A method of making a formulation that inhibits the onset or progression of liver fibrosis in a subject comprising:
providing a first population of hepatic stellate cells (HSCs);
measuring the amount of Transforming Growth Factor beta receptor III (TGFbr3) or an RNA encoding TGFBr3 in said first population of isolated HSCs;
providing a second population of HSCs;
providing a candidate agent;
contacting said second population of isolated HSCs with said candidate agent;
measuring the amount of TGFbr3 or an RNA encoding TGFbr3 in said second population of isolated HSCs after contact with said candidate agent;
comparing the amount of TGFbr3 or an RNA encoding TGFbr3 measured in said first isolated population of HSCs to the amount of TGFbr3 or a RNA encoding TGFbr3 measured in said second isolated population of HSCs after contact with said candidate agent;
selecting an agent that increases the amount of TGFbr3 or an RNA encoding TGFbr3 in said second isolated population of HSCs; and
formulating said agent for administration to a subject in need of an inhibition of the onset or progression of liver fibrosis.

31. A method of inhibiting the onset or progression of liver fibrosis in a subject comprising:
providing a first population of hepatic stellate cells (HSCs);
measuring the amount of Transforming Growth Factor beta receptor III (TGFbr3) or an RNA encoding TGFbr3 in said first population of isolated HSCs;
providing a second population of HSCs;
providing a candidate agent;
contacting said second population of isolated HSCs with said candidate agent;
measuring the amount of TGFbr3 or an RNA encoding TGFbr3 in said second population of isolated HSCs after contact with said candidate agent;
comparing the amount of TGFbr3 or an RNA encoding TGFbr3 measured in said first isolated population of HSCs to the amount of TGFbr3 or an RNA encoding TGFbr3 measured in said second isolated population of HSCs after contact with said candidate agent;
selecting an agent that increases the amount of TGFbr3 or an RNA encoding TGFbr3 in said second isolated population of HSCs;
providing said selected agent to a subject; and
measuring an inhibition of the onset or progression of liver fibrosis in said subject.

32. A method of identifying a predisposition for fibrosis in a tested subject comprising:
providing a biological sample comprising a population of hepatic stellate cells (HSCs) obtained from said tested subject;
isolating said population of HSCs from said biological sample;
measuring the amount of Transforming Growth Factor beta receptor III (TGFbr3) or an RNA encoding TGFbr3 in said isolated population of HSCs; and
identifying a predisposition for fibrosis in said tested subject when the amount of TGFbr3 or an RNA encoding TGFbr3 measured in said isolated population of HSCs is less than the standard amount of TGFbr3 or an RNA encoding TGFbr3 in HSCs from normal liver or less than or equal to the standard amount of TGFbr3 or an RNA encoding TGFbr3 in HSCs from fibrotic liver.

33. A method of generating a fibrosis ratio comprising:
providing a biological sample comprising a population of hepatic stellate cells (HSCs);
isolating said population of HSCs from said biological sample;
measuring the amount of Transforming Growth Factor beta receptor III (TGFbr3) or an RNA encoding TGFbr3 in said isolated population of HSCs;
measuring the amount of a housekeeping enzyme (e.g., GAPDH, actin beta, or ubiquitin) or an RNA encoding a housekeeping enzyme (e.g., GAPDH, actin beta, or ubiquitin) in said isolated population of HSCs; and
generating a fibrosis ratio that represents the amount of TGFbr3 or a RNA encoding TGFbr3 in relation to the amount of said housekeeping enzyme (e.g., GAPDH, actin beta, or ubiquitin) or an RNA encoding a housekeeping enzyme (e.g., GAPDH, actin beta, or ubiquitin) measured in said isolated population of HSCs.

34. A method of identifying a predisposition for fibrosis in a tested subject comprising:
providing a biological sample comprising a population of hepatic stellate cells (HSCs) obtained from said tested subject;
isolating said population of HSCs from said biological sample;
measuring the amount of Transforming Growth Factor beta receptor III (TGFbr3) or an RNA encoding TGFbr3 in said isolated population of HSCs;
measuring the amount of a housekeeping enzyme (e.g., GAPDH, actin beta, or ubiquitin) or an RNA encoding a housekeeping enzyme (e.g., GAPDH, actin beta, or ubiquitin) in said isolated population of HSCs;
generating a test subject fibrosis ratio that represents the amount of TGFbr3 or a RNA encoding TGFbr3 in relation to the amount of said housekeeping enzyme (e.g., GAPDH, actin beta, or ubiquitin) or an RNA encoding a housekeeping enzyme (e.g., GAPDH, actin beta, or ubiquitin) measured;
comparing said test subject fibrosis ratio to a standard normal fibrosis ratio that represents the amount of TGFbr3 or a RNA encoding TGFbr3 in relation to the amount of said housekeeping enzyme (e.g., GAPDH, actin beta, or ubiquitin) or an RNA encoding a housekeeping enzyme (e.g., GAPDH, actin beta, or ubiquitin) in HSCs from a normal liver; or
comparing said test subject fibrosis ratio to a standard abnormal fibrosis ratio that represents the amount of TGFbr3 or a RNA encoding TGFbr3 in relation to the amount of said housekeeping enzyme (e.g., GAPDH, actin beta, or ubiquitin) or an RNA encoding a housekeeping enzyme (e.g., GAPDH, actin beta, or ubiquitin) in HSCs from a fibrotic liver; and
identifying a predisposition for fibrosis in said tested subject when the tested fibrosis ratio is less than the standard normal fibrosis ratio or less than or equal to the standard abnormal fibrosis ratio.

35. A method of monitoring the progression of liver fibrosis or monitoring the efficacy of a treatment for liver fibrosis in a tested subject comprising:
providing a first biological sample comprising a population of hepatic stellate cells (HSCs) obtained from said tested subject at a first time point;
isolating said population of HSCs from said first biological sample;
measuring the amount of Transforming Growth Factor beta receptor III (TGFbr3) or an RNA encoding TGFbr3 in said isolated population of HSCs at said first time point;
measuring the amount of a housekeeping enzyme (e.g., GAPDH, actin beta, or ubiquitin) or an RNA encoding a housekeeping enzyme (e.g., GAPDH, actin beta, or ubiquitin) in said isolated population of HSCs at said first time point;
generating a first time point fibrosis ratio that represents the amount of TGFbr3 or an RNA encoding TGFbr3 in relation to the amount of said housekeeping enzyme (e.g., GAPDH, actin beta, or ubiquitin) or an RNA encoding a housekeeping enzyme (e.g., GAPDH, actin beta, or ubiquitin) measured at said first time point;
providing a second biological sample comprising a population of hepatic stellate cells (HSCs) obtained from said tested subject at a second time point;
isolating said population of HSCs from said second biological sample;
measuring the amount of Transforming Growth Factor beta receptor III (TGFbr3) or an RNA encoding TGFbr3 in said isolated population of HSCs at said second time point;
measuring the amount of a housekeeping enzyme (e.g., GAPDH, actin beta, or ubiquitin) or an RNA encoding a housekeeping enzyme (e.g., GAPDH, actin beta, or ubiquitin) in said isolated population of HSCs at said second time point;
generating a second time point fibrosis ratio that represents the amount of TGFbr3 or an RNA encoding TGFbr3 in relation to the amount of said housekeeping enzyme (e.g., GAPDH, actin beta, or ubiquitin) or an RNA encoding a housekeeping enzyme (e.g., GAPDH, actin beta, or ubiquitin) measured at said second time point; and
comparing said first and said second fibrosis ratios to determine the progression of liver fibrosis or efficacy of said treatment for liver fibrosis.

36. A method of identifying an agent that modulates the amount of Transforming Growth Factor beta receptor III (TGFbr3) in hepatic stellate cells (HSCs) comprising:
providing a plurality of cells comprising hepatic stellate cells (HSCs);
isolating a population of HSCs from said plurality of cells;
contacting said population of isolated HSCs or said plurality of cells with a candidate agent;
measuring the amount of Transforming Growth Factor beta receptor III (TGFbr3) or an RNA encoding TGFbr3 in said isolated population of HSCs after contact with said candidate agent;
measuring the amount of a housekeeping enzyme (e.g., GAPDH, actin beta, or ubiquitin) or an RNA encoding a housekeeping enzyme (e.g., GAPDH, actin beta, or ubiquitin) in said isolated population of HSCs after contact with said candidate agent;
generating a candidate agent fibrosis ratio that represents the amount of TGFbr3 or a RNA encoding TGFbr3 in relation to the amount of said housekeeping enzyme (e.g., GAPDH, actin beta, or ubiquitin) or an RNA encoding a housekeeping enzyme (e.g., GAPDH, actin beta, or ubiquitin) measured;
comparing said candidate agent fibrosis ratio to a standard normal fibrosis ratio that represents the amount of TGFbr3 or a RNA encoding TGFbr3 in relation to the amount of said housekeeping enzyme (e.g., GAPDH, actin beta, or ubiquitin) or an RNA encoding a housekeeping enzyme (e.g., GAPDH, actin beta, or ubiquitin) in HSCs from a normal liver when said plurality of cells comprising HSCs are obtained from normal liver; or
comparing said candidate subject fibrosis ratio to a standard abnormal fibrosis ratio that represents the amount of TGFbr3 or a RNA encoding TGFbr3 in relation to the amount of said housekeeping enzyme (e.g., GAPDH, actin beta, or ubiquitin) or an RNA encoding a housekeeping enzyme (e.g., GAPDH, actin beta, or ubiquitin) in HSCs from a fibrotic liver when said plurality of cells comprising HSCs are obtained from fibrotic liver; and
identifying said agent that modulates the amount of TGFbr3 or a RNA encoding TGbr3 in HSCs when the candidate agent fibrosis ratio differs from the standard normal fibrosis ratio when said plurality of cells comprising HSCs are obtained from normal liver or when the candidate agent fibrosis ratio differs from the abnormal fibrosis ratio when said plurality of cells comprising HSCs are obtained from fibrotic liver.
